SEQUENCE LISTING (138 sequences)

SEQ ID NO: 1 - protein, 94 aa, Homo sapiens
EKKEQKEKEKKEQEIKKKFKLTGPIQVIHLAKACCDVKGGKNELSFKQGEQIEIIRITDNPEGKWLGRTARGSYGYIKTTAVEIDYDSLKLKKD

SEQ ID NO: 2 - protein, 50 aa, Homo sapiens
ELVQKFQVYYLGNVPVAKPVGVDVINGALESVLSSSSREQWTPSHVSVAP

SEQ ID NO: 3 - protein, 50 aa, Homo sapiens
GLSLSVPATRQVIANHHMPSISFASGGDTDMTDYVAYVAKDPINQRACHI

SEQ ID NO: 4 - protein, 50 aa, Homo sapiens
MTLEELVACDNAAQKMQTVTAAVEELLVAAQRQDRLTVGVYESAKLMNVD

SEQ ID NO: 5 - protein, 53 aa, Homo sapiens
ENEEAEGDEIYEDLMRSEPVSMPPKMTEYDKRCCCLREIQQTEEKYTDTLLER

SEQ ID NO: 6 - protein, 50 aa, Homo sapiens
LEEEPSVETEDPSPETFRQLFRLFCYQEVAGPREALSRLWELCCRWLRPE

SEQ ID NO: 7 - protein, 37 aa, Homo sapiens
QELCRQLFRQFCYQDSPGPREALSRLRELCCQWLKPE

SEQ ID NO: 8 - protein, 37 aa, Homo sapiens
SEACRQRFRQFCYGDVHGPHEAFSQLWELCCRWLRPE

SEQ ID NO: 9 - protein, 39 aa, Homo sapiens
CEVFRQRFRQFQYREAAGPHEAFNKLWELCCQWLKPKPH

SEQ ID NO: 10 - protein, 49 aa, Homo sapiens
KGLEGMIRKRSGGHRVPGLTCCGRDQVCYRWSKRWLWKDSFLLYMCLET

SEQ ID NO: 11 - protein, 50 aa, Homo sapiens
KGIEGMIMKRSGGHRIPGLNCCGQGRACYRWSKRWLIVKDSFLLYMKPDS

SEQ ID NO: 12 - protein, 50 aa, Homo sapiens
ILRVGFVLRSARRAPCCRIMVLCSDRILFGHRGVNLDGNFFTVHAEFKLK

SEQ ID NO: 13 - protein, 49 aa, Homo sapiens
NTMCGYLKRKLRNSSGWQDLWWMCCHTLYFYRNHNEREPLAHLSLMDYG

SEQ ID NO: 14 - protein, 50 aa, Homo sapiens
GWKQCCVRYLLPWSKRWLMVRDSFVAYMDHRTEQIRMVLLMDRDFKVAAG

SEQ ID NO: 15 - protein, 50 aa, Homo sapiens
FIMEGTLTRVGAKHERHIFLFDGLMICCKSNHGQPRLPGASSAEYRLKEK

SEQ ID NO: 16 - protein, 49 aa, Homo sapiens
VLKQGYMMKKGHRRKNWTERWFVLKPNIISWVSEDLKDKKGDILLDENC

SEQ ID NO: 17 - protein, 50 aa, Homo sapiens
VLKAGYLEKRRKDHSFLGFEWQKRWCALSKTVFYYYGSDKDKQQKGEFAI

SEQ ID NO: 18 - protein, 50 aa, Homo sapiens
ALELGTVMTVFSARKSTPERRTVQMIMETRQVAWSKTADKIEGFLDIMEI

SEQ ID NO: 19 - protein, 49 aa, Homo sapiens
LQYFGQLLVWDWNVCKADIEREYHVYLFEKILLCCKEMSTLKRQARSIS

SEQ ID NO: 20 - protein, 50 aa, Homo sapiens
ALELGTVMTVFSFRKSTPERRTVQVIMETRQVAWSKTADKIEGFLDIMEI

SEQ ID NO: 21 - protein, 50 aa, Homo sapiens
FIMEGTLTRVGAKHERHIFLFDGLMICCKSNHGQPRLPGASNAEYRLKEK

SEQ ID NO: 22 - protein, 50 aa, Homo sapiens
YYKERLLYLEEGQKDSLIDSSRVLCCHGELKNNRGVKLHVFLFQEVLVIT

SEQ ID NO: 23 - protein, 50 aa, Homo sapiens
RLCEGTLFRKISSRRRQDKLWFCCLSPNHKLLQYGDMEEGASPPTLESLP

SEQ ID NO: 24 - protein, 50 aa, Homo sapiens
DTKHGMMKFREDRSLLGLGLPSGGFHDRYFILNSSCLRLYKEVRSQRPWS

SEQ ID NO: 25 - protein, 54 aa, Homo sapiens
LSMGDLLLHTTVIWLNPPASLGKWKKEPELAAFVFKTAWLVYKDGSKQKILWEQ

SEQ ID NO: 26 - protein, 50 aa, Homo sapiens
GVKLEVNERILGCCTSLMQAIQVLIVASKDLQREIVESGRGTASPKEFYA

SEQ ID NO: 27 - protein, 50 aa, Homo sapiens
CPSPVLNTPWIPFQNCCYNFIITKNRHMATTQDEVHTKCQKLNPKSHILS

SEQ ID NO: 28 - protein, 30 aa, Homo sapiens
EDARIMLSRAVECCPTSVELWLALARLETY

SEQ ID NO: 29 - protein, 50 aa, Homo sapiens
RFIFDVGTRLGLHYDTLATGIIYFHRFYMFHSFKQFPRYVTGACCLFLAG

SEQ ID NO: 30 - protein, 50 aa, Homo sapiens
NFLRRISKADDYDVQSRTLGKYLLEITIVDYKFIGMRPSLCCASAMYLAR

SEQ ID NO: 31 - protein, 50 aa, Homo sapiens
MQIIDVASILGLDCDISEHAFQLFRDCCSATCLRNRSVEALATACLVQAI

SEQ ID NO: 32 - protein, 50 aa, Homo sapiens
CFMRRFLKAAQSEKKLELLSFFMIELSLVEYEMLQFCPSMLAAAAIYTAQ

SEQ ID NO: 33 - protein, 50 aa, Homo sapiens
DFIMYCHTRLNLSTSTLFLTFTILDKYSSRFIIKSYNYQLLSLTALWISS

SEQ ID NO: 34 - protein, 50 aa, Homo sapiens
IRIRNLCERIQYMEQTERVYNVFKQILDQQTTLFFNRHMHQLILCCLYGV

SEQ ID NO: 35 - protein, 41 aa, Homo sapiens
MQREVWMSVFRYLSRRELCECMRVCKTWYKWCCDKRLWTKI

SEQ ID NO: 36 - protein, 50 aa, Homo sapiens
YSKWYFEVMVDEVTPFLTAQATHLRVGWALTEGYTPYPGAGEGWGGNGVG

SEQ ID NO: 37 - protein, 23 aa, Homo sapiens
WASTEGYSPYPGGGEEWGGNGVG

SEQ ID NO: 38 - protein, 50 aa, Homo sapiens
MAGKSSLFKVILLGDGGVGKSSLMNRYVTNKFDTQLFHTIGVEFLNKDLE

SEQ ID NO: 39 - protein, 50 aa, Homo sapiens
EFVAIADYAATDETQLSFLRGEKILILRQTTADWWWGERAGCCGYIPANH

SEQ ID NO: 40 - protein, 50 aa, Homo sapiens
HRTLLYGHAILLRHSYSGMYLCCLSTSRSSTDKLAFDVGLQEDTTGEACW

SEQ ID NO: 41 - protein, 50 aa, Homo sapiens
KMFVGQVPRTWSEKDLRELFEQYGAVYEINVLRDRSQNPPQSKGCCFVTF

SEQ ID NO: 42 - protein, 50 aa, Homo sapiens
RIIVENLFYPVTLDVLHQIFSKFGTVLKIITFTKNNQFQALLQYADPVSA

SEQ ID NO: 43 - protein, 50 aa, Homo sapiens
SVIGGNLTGIFIHRVTPGSAADQMALRPGTQIVMVDYEASEPLFKAVLED

SEQ ID NO: 44 - protein, 50 aa, Homo sapiens
HDFGFSVSDGLLEKGVYVHTVRPDGPAHRGGLQPFDRVLQVNHVRTRDFD

SEQ ID NO: 45 - protein, 50 aa, Homo sapiens
EDFGFSVADGLLEKGVYVKNIRPAGPGDLGGLKPYDRLLQVNHVRTRDFD

SEQ ID NO: 46 - protein, 48 aa, Homo sapiens
PAIGDICCAQFSEDDQWYRASVLAYASEESVLVGYVDYGNFEILSLMR

SEQ ID NO: 47 - protein, 50 aa, Homo sapiens
FKAEIGQPCCAFFAGDGSWYRALVKEILPNGHVKVHFVDYGNIEEVTADE

SEQ ID NO: 48 - protein, 49 aa, Homo sapiens
YRPRIGDACCAKYTSDDFWYRAVVLGTSDTDVEVLYADYGNIETLPLCR

SEQ ID NO: 49 - protein, 50 aa, Homo sapiens
KDKLKMEVDQLKKEVTLERMLVSKCCEEVRDYVEERSGEDPLVKGIPEDK

SEQ ID NO: 50 - protein, 50 aa, Homo sapiens
MDDREDLVYQAKLAEQAERYDEMVESMKKVAGMDVELTVEERNLLSVAYK

SEQ ID NO: 51 - protein, 50 aa, Homo sapiens
NTTLRCCLSPEENAEDMEVRWFQSQFSPAVFVYKGGRERTEEQKEEYRGR

SEQ ID NO: 52 - protein, 50 aa, Homo sapiens
SRAPHMIRDRKYHLKTYRQCCVGTELVDWMMQQTPCVHSRTQAVGMWQVL

SEQ ID NO: 53 - protein, 50 aa, Homo sapiens
ATLTILHQQTEAVLGECRVRFLSFLAVGRDVHTFAFIMAAGPASFCCHMF

SEQ ID NO: 54 - protein, 50 aa, Homo sapiens
LECCEGLAQSIISTVGQAFELRFKQYLHSPPKVALPPERLAGPEESAWGD

SEQ ID NO: 55 - protein, 49 aa, Homo sapiens
PDSWLCLLAIDEEEEDDIALQIHFTLIQSFCCDNDINIVRVSGMQRLAQ

SEQ ID NO: 56 - protein, 53 aa, Homo sapiens
GSIQQHFLKPLQRFLKPQDIEIIFINIEDLLRVHTHFLKEMKEALGTPGALER

SEQ ID NO: 57 - protein, 50 aa, Homo sapiens
LRTKEQILELLVLEQFLTVLPGEIQARVREQQPESGEEAVVLVEGLQRKP

SEQ ID NO: 58 - protein, 50 aa, Homo sapiens
IHTKEQILELLVLEQFLTILPGDLQAWVHEHYPESGEEAVTILEDLERGT

SEQ ID NO: 59 - protein, 50 aa, Homo sapiens
LRTKEQILELLVLEQFLTVLPGEIQGWVREQHPGSGEEAVALVEDLQKQP

SEQ ID NO: 60 - protein, 50 aa, Homo sapiens
MRSKEQILELLVLEQFLTILPTEIETWVREHCPENRERVVSLIEDLQREL

SEQ ID NO: 61 - protein, 50 aa, Homo sapiens
GAISFVQLFDPGFEVQVGKRSTEARHGVRIDTSHRSLILKCSSYRQARWW

SEQ ID NO: 62 - protein, 50 aa, Homo sapiens
GAIAFVLLVDKEFKIKVGKKETETKYGIRIDNLSRTLILKCNSYRHARWW

SEQ ID NO: 63 - protein, 50 aa, Homo sapiens
GMMIDEGDTYKVMDGENNVITLHNADISIVFAAPDRASWIEDITEAIKNA

SEQ ID NO: 64 - protein, 50 aa, Homo sapiens
VGLPTVADKIDNHENCFKLFYGSHTYFFRTDSYYFFERWVDSIFQAAISR

SEQ ID NO: 65 - protein, 50 aa, Homo sapiens
GKETEGIPTGLIITNSQHELHLKCRRLQDTATWKYIIEQAMGGIGNTWLQ

SEQ ID NO: 66 - protein, 50 aa, Homo sapiens
FFMRKVQINDKDDTSEYKHAFEIILKDGNSVIFSAKSAEEKNNWMAALIS

SEQ ID NO: 67 - protein, 48 aa, Homo sapiens
CVESLPDKDGKKCLFLVKCFDKTFEISASDKKKKQEWIQAIHSTIHLL

SEQ ID NO: 68 - protein, 50 aa, Homo sapiens
DGYSVRMNNTLRKDGKKDCCFEISAPDKRIYQFTAASPKDAEEWVQQLKF

SEQ ID NO: 69 - protein, 50 aa, Homo sapiens
KEIRPGKNSKDFERAKAVRHKADCCFTIFYGTQFVLSTLSLATDSKEDAV

SEQ ID NO: 70 - protein, 49 aa, Homo sapiens
MNKKTKRLDSLQLKGRILTSNITTWPNHHMGSYAIQIFWRGDPQHESFI

SEQ ID NO: 71 - protein, 50 aa, Homo sapiens
KEIRPGKNSKDFERAKAVRQKEDCCFTILYGTQFVLSTLSLAADSKEDAV

SEQ ID NO: 72 - protein, 50 aa, Homo sapiens
FFMRKVQINDKDDTNEYKHAFEIILKDENSVIFSAKSAEEKNNWMAALIS

SEQ ID NO: 73 - protein, 50 aa, Homo sapiens
RAVTHNEQLCYQLYRQPIPVKDLLLEDLQDGEVRLGGSLRGAFSNNERIK

SEQ ID NO: 74 - protein, 50 aa, Homo sapiens
EQLPVADMRALLTGKDCPHVREKGSGKQNKDLYELAFSISYDRGEEEAYL

SEQ ID NO: 75 - protein, 49 aa, Homo sapiens
GAPETSHRPEKEWPIKSLKVYLGVKKKLRPPTCWGFTWHETEKHEKQQW

SEQ ID NO: 76 - protein, 50 aa, Homo sapiens
KKLVGSHRLSIYEDWDPFRFRHMIPTEALQVRALASADAEANAVCEIVHV

SEQ ID NO: 77 - protein, 50 aa, Homo sapiens
KNSRWTEGLISASKAVGWGATVMVDAADLVVQGRGKFEELMVCSHEIAAS

SEQ ID NO: 78 - protein, 50 aa, Homo sapiens
IRDEKENNFVLEQLLYFNYMASWVMLGITYRNNSLMWFDKTPLSYTHWRA

SEQ ID NO: 79 - protein, 45 aa, Homo sapiens
KVEETPKKCKDIIKTARSLLNDVQFGQFGDDPKEEVMVLERILLQ

SEQ ID NO: 80 - protein, 34 aa, Homo sapiens
LILGKLPVWNGNLIHYSGGYRISDMRECIELMFQ

SEQ ID NO: 81 - protein, 32 aa, Homo sapiens
REAQEPRTLQEISIAANVQQKEIGKYIKILGE

SEQ ID NO: 82 - protein, 33 aa, Homo sapiens
CTINGFKSWNKCCELHTRYSEEHLMVCSRMMVE

SEQ ID NO: 83 - protein, 37 aa, Homo sapiens
KFWDSKNRMATLKVLQNLCCNQYSIKQFTTMEMHLFK

SEQ ID NO: 84 - protein, 50 aa, Homo sapiens
AKVCQLELSFREILNNYKKEAQCKPEVFLSIYIGSRNHNGVLISRHVDII

SEQ ID NO: 85 - protein, 50 aa, Homo sapiens
DDLYSYGFDGLHLWTGHVARPVTSPGQHLLAPEDVISCCLDLSVPSISFR

SEQ ID NO: 86 - protein, 50 aa, Homo sapiens
DDLFSYGFDGLHLWSGCIARTVSSPNQHLLRTDDVISCCLDLSAPSISFR

SEQ ID NO: 87 - protein, 50 aa, Homo sapiens
VDGHFVTMQIWDTAGQERFRSLRTPFYRGSDCCLLTFSVDDSQSFQNLSN

SEQ ID NO: 88 - protein, 5 aa, Homo sapiens
VGKHV

SEQ ID NO: 89 - protein, 6 aa, Homo sapiens
WTIHPA

SEQ ID NO: 90 - protein, 29 aa, Homo sapiens
YTRKAALEAQNALHNMKVLPGMHHPIQMK

SEQ ID NO: 91 - protein, 20 aa, Homo sapiens
QHAKLSLDGQNIYNACCTLR

SEQ ID NO: 92 - protein, 25 aa, Homo sapiens
TTLEEAVGLLRRVDGFCCLSVKVNT

SEQ ID NO: 93 - protein, 23 aa, Homo sapiens
CCLAVPLLAEAGDVLELIISRKP

SEQ ID NO: 94 - protein, 23 aa, Homo sapiens
CCLVVPLIAESGNKLDLVISRNP

SEQ ID NO: 95 - protein, 9 aa, Homo sapiens
LCPIIPKLL

SEQ ID NO: 96 - protein, 9 aa, Homo sapiens
LRMISSTFL

SEQ ID NO: 97 - protein, 9 aa, Homo sapiens
VQPITSSHL

SEQ ID NO: 98 - protein, 13 aa, Homo sapiens
NPFKELKGGCVIS

SEQ ID NO: 99 - protein, 50 aa, Homo sapiens
NVIGARRASWRIISSIEQKEENKGGEDKLKMIREYRQMVETELKLICCDI

SEQ ID NO: 100 - protein, 31 aa, Homo sapiens
TTFVSKDSRGSVALIIHNVTAEDNGIYQCYF

SEQ ID NO: 101 - protein, 26 aa, Homo sapiens
LEDGVLNHVDQEHHFQDKYLFYRFLD

SEQ ID NO: 102 - protein, 30 aa, Homo sapiens
WCEPNAASLSEAVQAACMLRYQKCLDARSQ

SEQ ID NO: 103 - protein, 50 aa, Homo sapiens
EEDSLEHNYYNSIPGKEPPLGGLVDSRLALTQPCALTALDQGPSPSLRDA

SEQ ID NO: 104 - protein, 50 aa, Homo sapiens
LLGEPAETQGTTEARDLHCLLVTMPHTDAWKSHGLVEVASYCEESRGNNQ

SEQ ID NO: 105 - protein, 50 aa, Homo sapiens
ANLYQVFIKYKERFLVYGRYCSQVESASKHLDRVAAAREDVQMKLEECSQ

SEQ ID NO: 106 - protein, 7 aa, Homo sapiens
RKHRQRG

SEQ ID NO: 107 - protein, 26 aa, Homo sapiens
DEAVLQVQAHEHGQEIFQKKVSPPGP

SEQ ID NO: 108 - protein, 18 aa, Homo sapiens
VKAWRQDVPSEEAEPEAA

SEQ ID NO: 109 - protein, 22 aa, Homo sapiens
EIPEQQVDMHDILLEELAPVGT

SEQ ID NO: 110 - protein, 11 aa, Homo sapiens
AQEITELAQGP

SEQ ID NO: 111 - protein, 11 aa, Homo sapiens
GGAIEEFIQKH

SEQ ID NO: 112 - protein, 11 aa, Homo sapiens
PHRFSSSFPVR

SEQ ID NO: 113 - protein, 5 aa, Homo sapiens
LQYRS

SEQ ID NO: 114 - protein, 5 aa, Homo sapiens
VLQDM

SEQ ID NO: 115 - protein, 13 aa, Homo sapiens
KWLSGLKILHQEA

SEQ ID NO: 116 - protein, 23 aa, Homo sapiens
LKLRNEESHKLWMSVLNRLLWKN

SEQ ID NO: 117 - protein, 13 aa, Homo sapiens
NWLSGLKILHQEA

SEQ ID NO: 118 - protein, 5 aa, Homo sapiens
LQYRS

SEQ ID NO: 119 - protein, 40 aa, Homo sapiens
NFFRVSFKNGSQSQTHSLQANDTFNKQQWLNCIRQAKETV

SEQ ID NO: 120 - protein, 24 aa, Homo sapiens
NFIAPSKREFYLWTDGLSALLGSP

SEQ ID NO: 121 - protein, 24 aa, Homo sapiens
YLCCDTQMELREWFATFLFVQHDG

SEQ ID NO: 122 - protein, 36 aa, Homo sapiens
KSESEGRPERVFHLCCSSPESRKDFLKAVHSILRDK

SEQ ID NO: 123 - protein, 49 aa, Homo sapiens
TAQLVAASKVKADKDSPNLAQLQQASRGVNQATAGVVASTISGKQIEET

SEQ ID NO: 124 - protein, 43 aa, Homo sapiens
GRPTIKNEKFLAGLSTDGFWDIQTFKVIEEAVYFHQHSILACK

SEQ ID NO: 125 - protein, 8 aa, Homo sapiens
TFYNEVFV

SEQ ID NO: 126 - protein, 38 aa, Homo sapiens
INGCPVQGVFESFNLDGLFFPVVSFSAGVKVRFLLGGH

SEQ ID NO: 127 - protein, 39 aa, Homo sapiens
INGQPVQGMFENFNIDGLFFPVVSFSAGIKVRFLLGGRH

SEQ ID NO: 128 - protein, 50 aa, Homo sapiens
WKKEFIYYADVKEPESFPFVILGNKIDISERQVSTEEAQAWCRDNGDYPY

SEQ ID NO: 129 - protein, 50 aa, Homo sapiens
LDVLDKHLIPAANTGESKVFYYKMKGDYHRYLAEFATGNDRKEAAENSLV

SEQ ID NO: 130 - protein, 10 aa, Homo sapiens
WVPYISLQER

SEQ ID NO: 131 - protein, 50 aa, Homo sapiens
RANNGRFTLRDLLMVPMQRVLKYHLLLQELVKHTQEAMEKENLRLALDAM

SEQ ID NO: 132 - protein, 14 aa, Homo sapiens
CSLPWDVGSTGTAP

SEQ ID NO: 133 - protein, 49 aa, Homo sapiens
DNMDFSSMTLTQIKRQEMDSQVRVLELENELQKERQKLGELRKKHYELA

SEQ ID NO: 134 - protein, 25 aa, Homo sapiens
FETSAKDATNVAAAFEEAVRRVLAT

SEQ ID NO: 135 - protein, 50 aa, Homo sapiens
AYKAASDIAMTELPPTHPIRLGLALNFSVFYYEILNSPDRACRLAKAAFD

SEQ ID NO: 136 - protein, 17 aa, Homo sapiens
RDLAQCVNEVKRDNETL

SEQ ID NO: 137 - protein, 45 aa, Homo sapiens
DAIAELDTLSEESYKDSTLIMQLLRDNLTLWTSDMQGDGEEQNKE

SEQ ID NO: 138 - protein, 82 aa, Homo sapiens
SFSHLEGLLQEAGPSEACCVRDVTEPGALRMETGDPITVIEGSSSFHSPDSTIWKGQNGRTFKVGSFPASAVTLADAGGLPA

(19) United States
(12) Patent Application Publication
(10) Pub. No.: US 2009/0087445 A1
(43) Pub. Date: Apr. 2, 2009

(54) Method for the Redox Potential-Dependent Detection of Target Molecules by Interacting Polypeptides

(75) Inventors: Christian Freund, Berlin (DE); Jurgen Zimmermann, Jamaica Plain, MA (US)

Correspondence Address: JOYCE VON NATZMER;PEQUIGNOT + MYERS LLC, 200 Madison Avenue, Suite 1901, New York, NY, 10016, US

(73) Assignee: FORSCHUNGSVERBUND BERLIN E.V., Berlin (DE)

(21) Appl. No.: 11/817,099

(22) PCT Filed: Feb. 24, 2006
(86) PCT No.: PCT/EP06/01960

§ 371 Date: Oct. 14, 2008

(30) Foreign Application Priority Data
Feb. 25, 2005 (EP) 05090045.5

(51) Int. Cl. A61K 39/00; C07K 7/06; C07K 7/08; C07K 14/435; C40B 40/10; C12N 15/01; A61K 38/08; A61K 38/17; G01N 33/558; A61K 38/10; C40B 30/04; C07H 21/04; C07H 21/02; C12N 15/63; C12N 5/00

(52) U.S. Cl. 424/185.1; 530/324; 530/326; 530/330; 530/329; 530/325; 530/328; 530/327; 506/18; 536/23.5; 536/24.5; 435/320.1; 435/325; 435/441; 506/9; 514/12; 514/13; 514/14; 514/15; 514/16; 514/17; 436/501

(57) ABSTRACT

The invention primarily relates to an amino acid sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD (=SEQ ID NO 1), a protein recognition domain comprising said amino acid sequence, the use of said amino acid sequence or protein recognition domain for identifying redox-dependent protein-protein or protein-lipid interactions, and to a method for the redox potential dependent detection of target molecules; the invention also relates to the use of said amino acid sequence as a marker for measuring the redox potential in cells.

The invention primarily relates to an amino acid sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD (=SEQ ID NO 1), a protein recognition domain comprising said amino acid sequence, the use of said amino acid sequence or protein recognition domain for identifying redox-dependent protein-protein or protein-lipid interactions, and to a method for the redox potential-dependent detection of target molecules (molecules bound by SEQ ID NO 1, the identification SEQ ID NO 1 referring to the amino acid sequence mentioned above); the invention also relates to the use of said amino acid sequence as a marker for measuring the redox potential in cells; furthermore, the invention relates to the use of said amino acid sequence for the transport and redox-dependent release of molecules bound to the side chain of the cysteines of SEQ ID NO 1; the invention also relates to the use of said amino acid sequence for establishing an amino acid sequence/polypeptide library; moreover, the invention relates to a kit comprising said amino acid sequence or said protein recognition domain; in addition, the invention relates to antibodies against amino acid sequences of SEQ ID NO 1 or of said polypeptide library and to the use of said amino acid sequence in pharmaceutical formulations. Apart from the preferred amino acid sequence, the invention also relates to the other preferred amino acid sequences SEQ ID NO 2 to SEQ ID NO 138.

Oxidative stress and changes in the redox equilibrium in organisms, especially in cells, are important indicators and essential risk factors in numerous processes of disease and ageing. Thus, for example, it has been described that reactive oxygen species play an important role in neurodegenerative diseases determined by age, such as Alzheimer's disease. Furthermore, many anti inflammatory medicaments used e.g. for autoimmune diseases are known to act as antioxidants. Chronic inflammatory diseases are frequently associated with a dysfunction of redox-dependent processes, e.g. rheumatoid arthritis, dermal diseases such as psoriasis and skin cancer, multiple sclerosis, tuberculosis, and chronic pulmonary and respiratory diseases such as asthma pneumoconiosis, nasal polyps and pulmonary fibrosis.

Furthermore, as disclosed in the prior art, many tumors comprise a pro-oxidative environment protecting degenerate cells from extracellular cell death signals.

Systemic oxidative stress also occurs in HIV-infected individuals. Redox control therefore represents an important therapeutic strategy in persons suffering from HIV. Oxidative stress is also an indicator in chronic renal diseases.

It follows from this that redox sensitive inhibitors could be used as pharmaceutical agents for diagnosis or therapy in all the above-mentioned diseases, provided, they were available. However, despite a wide variety of examples demonstrating the importance of redox processes in pathologic processes, medicaments having a specific effect, being sensitive in their effect to the redox state of a tissue and taking effect under pathologic redox conditions only, are unavailable as yet. This is due to currently lacking knowledge and characterization of components involved in redox signaling, but also to the difficulty of specifically inhibiting redox dependent macromolecules such as peptides, proteins, as well as lipid or carbohydrate structures.

The object of the invention was therefore to provide a polypeptide or an amino acid sequence which allows detection of target molecules whose inhibition is switchable in dependence on the redox potential, and which can be used for the identification of redox dependent protein-protein or protein-lipid interactions; another object of the invention was to provide probes or markers undergoing specific interaction with the target molecules.

The invention solves this problem by providing a polypeptide comprising a Cys-Cys motif having a reversibly adjustable redox potential in the range of from −400 mV to +200 mV, preferably in the range of from −300 mV to 0 mV, more preferably in the range of from 300 mV to 150 mV, which can be used particularly for detecting redox-dependent protein-protein or protein-lipid interactions and/or for detecting molecules which can be bound by the polypeptide in dependence on the redox potential, selected from the group consisting of the amino acid sequences in accordance with SEQ ID NO 1 to SEQ ID NO 138. Accordingly, the invention is directed particularly to the disclosed peptides or polypeptides or amino acid sequences and to the use thereof. The above mentioned possible uses are less in tended to disclose materials for a specified purpose but, in the first place, a single general inventive idea creating a technical relationship between the disclosed peptides. The disclosed peptides have properties or effects in common. The functional relationship is the reversibly adjustable redox potential in the range of from −400 mV to +200 mV which can be utilized especially in the detection of redox-dependent protein-protein or protein-lipid interactions and/or in the detection of molecules that are bound by the polypeptide in dependence on the redox potential. What is claimed in addition is, of course, the use of the peptides in medicine (purpose-limited product claim relating to an initial medical indication) and the use in research and other medical applications.

In a preferred embodiment the polypeptide is selected from the group consisting of SEQ ID NO 1 to SEQ ID NO 5.

In a preferred embodiment the polypeptide is selected from the group consisting of SEQ ID NO 5 to SEQ ID NO 10.

In a preferred embodiment the polypeptide is selected from the group consisting of SEQ ID NO 10 to SEQ ID NO 15.

In a preferred embodiment the polypeptide is selected from the group consisting of SEQ ID NO 15 to SEQ ID NO 20.

In a preferred embodiment the polypeptide is selected from the group consisting of SEQ ID NO 20 to SEQ ID NO 25.

In a preferred embodiment the polypeptide is selected from the group consisting of SEQ ID NO 25 to SEQ ID NO 30.

In a preferred embodiment the polypeptide is selected from the group consisting of SEQ ID NO 30 to SEQ ID NO 35.

In a preferred embodiment the polypeptide is selected from the group consisting of SEQ ID NO 35 to SEQ ID NO 40.

In a preferred embodiment the polypeptide is selected from the group consisting of SEQ ID NO 40 to SEQ ID NO 45.

In a preferred embodiment the polypeptide is selected from the group consisting of SEQ ID NO 45 to SEQ ID NO 50.

In a preferred embodiment the polypeptide is selected from the group consisting of SEQ ID NO 50 to SEQ ID NO 55.

In a preferred embodiment the polypeptide is selected from the group consisting of SEQ ID NO 60 to SEQ ID NO 65.

In a preferred embodiment the polypeptide is selected from the group consisting of SEQ ID NO 65 to SEQ ID NO 70.

In a preferred embodiment the polypeptide is selected from the group consisting of SEQ ID NO 70 to SEQ ID NO 75.

In a preferred embodiment the polypeptide is selected from the group consisting of SEQ ID NO 75 to SEQ ID NO 80.

In a preferred embodiment the polypeptide is selected from the group consisting of SEQ ID NO 80 to SEQ NO 85.

In a preferred embodiment the polypeptide is selected from the group consisting of SEQ ID NO 85 to SEQ ID NO 90.

In a preferred embodiment the polypeptide is selected from the group consisting of SEQ ID NO 90 to SEQ ID NO 95.

In a preferred embodiment the polypeptide is selected from the group consisting of SEQ ID NO 95 to SEQ ID NO 100.

In a preferred embodiment the polypeptide is selected from the group consisting of SEQ ID NO 100 to SEQ ID NO 105.

In a preferred embodiment the polypeptide is selected from the group consisting of SEQ ID NO 105 to SEQ ID NO 110.

In a preferred embodiment the polypeptide is selected from the group consisting of SEQ ID NO 110 to SEQ ID NO 115.

In a preferred embodiment the polypeptide is selected from the group consisting of SEQ ID NO 115 to SEQ ID NO 120.

In a preferred embodiment the polypeptide is selected from the group consisting of SEQ ID NO 120 to SEQ ID NO 125.

In a preferred embodiment the polypeptide is selected from the group consisting of SEQ ID NO 125 to SEQ ID NO 130.

In a preferred embodiment the polypeptide is selected from the group consisting of SEQ ID NO 130 to SEQ ID NO 135.

In a preferred embodiment the polypeptide is selected from the group consisting of SEQ ID NO 135 to SEQ ID NO 138.

Each one of the above groups has preferred properties for solving the problem according to the invention.

As far as the description discloses the sequence SEQ ID NO 1, this not only refers to that preferred embodiment because, as will be appreciated, quoting this sequence also includes the other preferred sequences SEQ ID NO 2 to SEQ ID NO 138. Accordingly, SEQ ID NO 1 also represents SEQ ID NO 1 to SEQ ID NO 138 in each case, especially in the preferred groups of sequences mentioned above.

In a preferred embodiment the invention solves the above-mentioned problem preferably by providing an amino acid sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD (SEQ ID NO 1) or an amino acid sequence SF SHLEGLLQEA GPSEACCVRD VTEPGALRME TGDPITVIEG SSSFHSPDST IWKGQNGRTF KVGSFPASAV TLADAGGLPA (SEQ ID NO 138) or a nucleic acid sequence encoding the above-mentioned amino acid sequence.

The nucleic acid molecule of the invention is selected from the group consisting of:
a) a nucleic acid molecule encoding an amino acid sequence according to SEQ ID NO 1 or SEQ ID NO 1 to SEQ ID NO 138, or complementary nucleotide sequences thereof,
b) a nucleic acid molecule hybridizing with a nucleotide sequence according to a) under stringent conditions,
c) a nucleic acid molecule comprising a nucleotide sequence having sufficient homology to be functionally analogous to a nucleotide sequence according to a),
d) a nucleic acid molecule which, as a consequence of the genetic code, is degenerated into a nucleotide sequence according to a), and
e) a nucleic acid molecule in accordance with a nucleotide sequence according to a), which is modified and functionally analogous to a nucleotide sequence according to a) as a result of deletions, additions, substitutions, translocations, inversions and/or insertions.

Advantageous embodiments of the invention are presented in the subclaims.

Advantageously, the structure of the nucleic acid sequence is such that an amino acid sequence is encoded which consists of the sequence in accordance with SEQ ID NO 1 or SEQ ID NO 2 to SEQ ID NO 138, especially in accordance with the above-mentioned groups.

With reference to some advantageous embodiments and various aspects of the technical teaching of the invention, the invention will be explained in more detail below. Following a description of the products according to the invention, i.e., the molecules which, synonymously, may also be referred to as amino acid sequences, peptides or proteins or mixtures thereof, preferred inventive methods and applications will be described: (i) initially, the amino acid sequence of the invention, SEQ ID NO 1, (ii) the spatial structure of the amino acid sequence in accordance with SEQ ID NO 1, and (iii) recognition molecules directed against the latter will be illustrated in more detail, followed by (iv) a description of the production methods of the amino acid sequence in accordance with SEQ ID NO 1; (v) subsequently, methods of obtaining preferred modifications and variants (muteins) of the amino acid sequence according to SEQ ID NO 1 will be disclosed, as well as (vi) a method of detecting and obtaining modified amino acid sequences in accordance with SEQ ID NO 1 which bind to well-defined target molecules; and finally (vii), advantageous uses of the amino acid sequence in accordance with SEQ ID NO 1 and of muteins derived therefrom, as well as (viii) further advantages of the teaching according to the invention will be explained.

(i) The sequence according to the invention, selected from the group consisting of SEQ ID NO 1 to SEQ ID NO 138: in a preferred fashion the nucleic acid molecule essentially encodes the amino acid sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD (=SEQ ID NO 1) or a functionally analogous structure such as SF SHLEGLLQEA GPSEACCVRD VTEPGALRME TGDPITVIEG SSSFHSPDST IWKGQNGRTF KVGSFPASAV TLADAGGLPA. In the meaning of the invention, the term “essentially” (or functionally analogous structure) implies that the amino acid sequence, in particular, consists of the sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD, it being possible, however, that the sequence comprises an additional amino acid or another structure (lipid and/or sugar residues). Although the material according to the invention consists of the amino acid sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD, it will be appreciated by those skilled in the art that modifications by additions, deletions or substitutions can be made without substantially changing the polypeptide. The modified amino acid sequence is not substantially changed if it achieves the same function as the sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD essentially in the same way, leading to the same result. In the meaning of the invention this implies that the modified amino acid sequence (=amino acid sequence essentially corresponding to the amino acid sequences SEQ ID NO 1 to SEQ ID NO 138 according to the invention) essentially corresponds to the sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD (SEQ ID NO 1), in particular, if the use thereof has no substantial effect on the solution of the problem according to the invention and if this is obvious to a person skilled in the art, and it will be appreciated by those skilled in the art that the inventors do not intend to restrict their teaching to the wording of the claims, i.e., to the sequences in accordance with SEQ ID NO 1 to SEQ ID NO 138.

In a preferred embodiment of the invention the nucleic acid molecule having sufficient homology to be functionally analogous to a nucleotide sequence encoding an amino acid sequence in accordance with SEQ ID NO 1 or to a complementary nucleotide sequence thereof has at least 40% homology. In the meaning of the invention, functional analogy to the above-mentioned nucleic acid sequences or to sequences hybridizing with said nucleic acid sequences implies that the homologous nucleic acid sequences, as well as the encoded amino acid sequences, exhibit a behavior allowing conclusions as to a target molecule whose inhibition is switchable in dependence on the redox potential.

In another advantageous embodiment of the invention, the nucleic acid molecule has at least 60%, preferably 70%, more preferably 80%, and most preferably 90% homology to a nucleic acid molecule encoding an amino acid sequence in accordance with SEQ ID NO 1 or to complementary nucleotide sequences thereof, said nucleic acid molecule having a biological activity just like the nucleic acid molecule encoding a sequence in accordance with SEQ ID NO 1 or a complementary sequence thereof. In another preferred embodiment of the invention the nucleic acid molecule is a genomic DNA, a cDNA and/or an RNA. The invention also relates to vectors and host cells and/or vaccines comprising the nucleic acid molecule according to the invention.

The amino acid sequence or the polypeptide in accordance with SEQ ID NO 1 (according to the above=SEQ ID NO 1 to SEQ ID NO 138) shows surprising properties with respect to its behavior under either reducing or oxidizing conditions. More specifically, NMR-spectroscopic investigations have demonstrated that the two neighboring cysteines in SEQ ID NO 1 can reversibly form an intramolecular disulfide bridge. In both forms, the polypeptide or protein is present as monomer, the structure of the amino acid sequence defined by SEQ ID NO 1 undergoing significant changes under reducing or oxidizing conditions. Using dithiothreitol (reducing) or hydrogen peroxide (oxidizing), the redox state can be established unambiguously because no cysteines in addition to the neighboring cysteines are present in SEQ ID NO 1. Reduced and oxidized glutathione, an intracellular redox buffer, is also suitable in adjusting one of the two conformations, and any redox buffer system is possible to adjust the redox equilibrium of the amino acid sequence according to SEQ ID NO 1. Advantageous properties of the amino acid sequence according to SEQ ID NO 1 to SEQ ID NO 138, particularly SEQ ID NO 1, are high solubility, relatively high thermal stability, and low tendency of aggregation.

The invention is also directed to a protein recognition domain comprising said amino acid sequence or mutants or muteins derived therefrom. Using standard procedures of biochemistry or genetics, the protein recognition domain can be converted into functionally analogous derivatives and structurally homologous macromolecules in such a way that those whose inhibition can be modified in dependence on the redox potential undergo particularly efficient interaction with target molecules. Targets in the meaning of the invention are e.g. sugars, RNA, DNA, amino acids, vitamins, second messengers, particularly proteins or lipids.

(ii) Structure of the amino acid sequence according to SEQ ID NO 1: as a result of structure elucidation, a domain has been identified which belongs to a family of protein interaction domains. Advantageously, the domain can be modified by suitable mutations in such a way that it can be used as binding partner for some other target molecule. Surprisingly, it has been demonstrated that such binding is dependent on the redox potential (of the environment). As a consequence, it is possible to develop specific target molecules of a particular variant of the domain which bind to the target molecule in the one redox form but not in the other.

The three-dimensional structure of the domain or amino acid sequence according to SEQ ID NO 1 was elucidated by means of NMR-spectroscopic methods. It was found that the domain can be reversibly converted into an oxidized or reduced form using H₂O₂ and dithiothreitol, respectively. Both forms are clearly different in their structure. The structural conversion is based on an oxidation or reduction of the two cysteines in the sequence. It is this structural conversion which represents an important aspect of the invention. Therefore, the invention also relates to peptide structures having at least two neighboring cysteines which can undergo redox-dependent conversion.

Using analytic ultracentrifugation and NMR methods, it was possible to demonstrate that both the oxidized form and reduced form of the domain, i.e. of the amino acid sequence according to SEQ ID NO 1, are present as monomers. Hence, an intramolecular reaction is involved wherein a disulfide bridge between the two neighboring cysteines is formed, and in a preferred fashion there is no formation of dimers etc. as a result of intermolecular disulfide bridge formation. However, the disulfide bridge can be a component of a specific protein interaction surface with another polypeptide. The domain binds to sequences in other proteins and to acid lipids such as those occurring in biological membranes. Each of the different domains of the peptides according to the invention is specific to particular sequences in its binding, comparable to the specificity of a particular antibody.

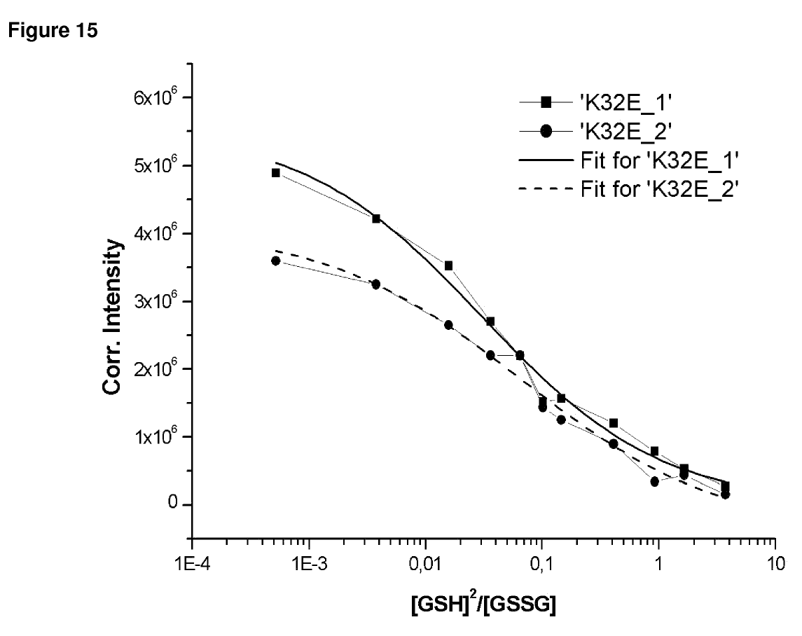

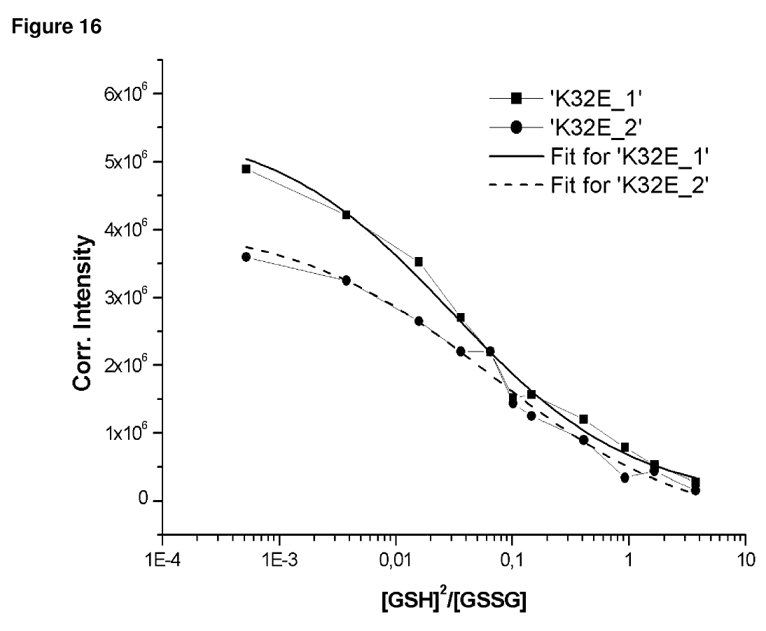

The structures of the reduced and oxidized forms of the amino acid sequences are based on NOE restraints summarized in Table 3. The characteristic feature of the structure is an N-terminal α-helix which is packed against a β-sheet and interacts via various hydrophobic contacts. More specifically, two cysteines are in direct neighborhood in positions 34 and 35 of the structure of the amino acid sequence in accordance with SEQ TD NO 1 and may form an intramolecular disulfide bridge in the oxidized variant. This property and the changes in the spatial arrangement of atoms as a result of the cysteinyl-cysteine ring closure are disclosed with reference to the two three dimensional structures which likewise represent a subject matter of this invention. Another aspect of the invention is the ensemble of the 3-dimensional coordinates (see examples). Electronic representations of these coordinates or schematic representations including the secondary structure elements and the profile of the chain (backbone conformation) represent another essential aspect of the invention. This also relates to the electronic representation of the structure of a mutein having a three-dimensional folding similar to the structure of the polypeptide in accordance with SEQ ID NO 1. Advantageously, the structures according to the invention allow the use of the method of rational mutagenesis in order to change the redox potential of the domain. Especially, but not exclusively, the positions 18, 20, 32, 38 to 43, 47, 48, 50, 59 to 64, 78, 79, 80 and/or 81, particularly 32, 48, according to the structure of the invention are replaced with one of the twenty natural amino acids well-known to those skilled in the art; the redox potential can be determined using e.g. NMR spectroscopy (as mentioned in the examples). That is, each one of the positions 32, 38 to 43, 47, 48, 50, 59 to 64, 78, 79, 80 and/or 81 can be occupied by the following amino acids: Gly, Ala, Val, Leu, Ile, Phe, Tyr, Trp, Pro, Asp, Glu, Asn, Gln, Ser, Thr, Cys, Met, Lys, Arg or His. In this way, variants of the protein according to the invention are generated which have a specific redox potential and can be used as scaffold proteins (see FIGS. 15 and 16 and section of examples).

The structure according to the invention also serves as a basis for introducing mutations resulting in larger cavities, using e.g. the VOIDOO program (Uppsala Software Factory). This allows specific introduction of binding pockets into the protein, e.g. for fluorophores, xenon or lipids. Furthermore, knowledge of the structure permits the use of a method for randomizing amino acids, which forms the basis of new binding properties of the sequences according to SEQ ID NO 1 or muteins. Also, the structure according to the invention advantageously allows to find small organic molecules binding to specific surface structures (such as hydrophobic pockets or regions of positive or negative electrostatic charge).

(iii) Preferred modifications and adaptations of the amino acid sequence in accordance with SEQ ID NO 1 to SEQ ID NO 138, preferably SEQ ID NO 1: of course, specific adaptations and modifications of the amino acid sequence can be made in order to obtain improved function. In the meaning of the invention, amino acid sequences modified in such a specific fashion are referred to as muteins (see above). The amino acid sequence of the invention, or the muteins thereof, can be further modified by suitable substitution, deletion or addition of further amino acids or by incorporating a glycoside residue without substantially impairing the capability of the amino acid sequence of interacting with a target molecule whose inhibition is switchable in dependence on the redox potential. In the meaning of the invention, such amino acid sequences, especially muteins, are also referred to as functionally analogous derivatives or structural homologues. However, non-switchable inhibition by muteins may also be preferred.

In particular, the muteins can be used as redox-sensitive inhibitors. Each polypeptide derived from the amino acid sequence of the invention (preferably SEQ ID NO 1) and binding to the target molecules in oxidized or reduced state only is a mutein in the meaning of the invention. The redox state of the inhibitor is sensitive to the actual environment. It will be appreciated that redox switchability is not a necessary property of the muteins. The muteins may also be binding variants which bind disease-related targets in a redox-independent fashion (i.e., both in reduced form and oxidized form). Such muteins—having redox independent recognition—are also preferred markers or analytic probes or therapeutic agents for the treatment of the above-mentioned diseases.

The muteins may also be referred to as functionally analogous sequences. Functionally analogous sequences in the meaning of the invention are those sequences which can be identified as having the same effect by a person skilled in the art. Having knowledge of nucleic acid molecules found in humans, a person skilled in the art may of course detect analogues and homologues in test animals as a result of homology or analogy investigations, by means of which redox processes can be investigated.

More specifically, “functionally analogous” means that the modified structural homologues or derivatives—just like the amino acid sequence in accordance with SEQ ID NO 1—have the capability of interacting with target molecules in a measurable way, or can be used for identifying redox-dependent protein-protein or protein-lipid interactions. That is to say, substitution, deletion or addition of amino acids, or adding or varying sugar residues will not impair the amino acid sequences or muteins in such a way that their binding behavior would be reduced to such an extent that the objects of the invention could no longer be solved.

For example, amino acids can be substituted in the amino acid sequence according to SEQ ID NO 1 on the basis of various criteria, including hydrophobicity, charge properties, polarity, size, presence of a functional group (e.g. NH₂ group, aromatic character). Assigning the amino acids to specific groups will easily be possible for a skilled specialist, and further suitable amino acid substitutions can be found e.g. in Bowie et al. (Science 247; 1306-1310 (1990)). For example, conservative amino acid substitutions can be made in amino acids having related side chains. Thus, natural amino acids are divided in four groups wherein amino acid substitutions are preferred especially in the sequence EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD or SF SHLEGLLQEA GPSEACCVRD VTEPGALRME TGDPITVIEG SSSFHSPDST IWKGQNGRTF KVGSFPASAV TLADAGGLPA: (1) acidic amino acids: aspartate and giutatmate; (2) basic amino acids: lysine, arginine and histidine; (3) non-polar: alanine, valine, leucine, isoleucine, proline, phenylalanine, methionine, tryptophan; (4) uncharged polar amino acids: glycine, asparagine, glutamine, cysteine, serine, threonine and tyrosine. Phenylalanine, tryptophan and tyrosine are sometimes defined as a group of amino acids having aromatic side chains. For example, it can be expected that an isolated replacement of leucine with isoleucine, of aspartate with glutamate, of threonine with serine or similar conservative substitutions will not result in a substantial change in activity or binding properties of the sequence in accordance with SEQ TD NO 1.

Preferred amino acid sequences in the meaning of the invention, especially peptides or proteins, are those including a Cys-Cys motif having a reversibly adjustable redox potential in the range of from −400 mV to +200 mV, preferably in the range of from −300 mV to 0 mV, more preferably in the range of from −300 mV to −150 mV, i.e., all those proteins having a Cys-Cys motif as disclosed in accordance with SEQ TD NO 1 to SEQ ID NO 138. In a preferred fashion the above peptides can be substituted by one of the 20 naturally occurring amino acids in directly neighboring positions w-x-Cys-Cys-y-z (w, x, y, z: any of the twenty natural amino acids) and may have a modified redox potential of values between −400 and +200 mV.

(iv) Recognition molecules directed against the molecules according to the invention: The invention also relates to a recognition molecule directed against said nucleic acid molecule, said vector, said host cell and/or said polypeptide, i.e., the amino acid sequence. Recognition substances in the meaning of the invention are molecules capable of interacting with the above mentioned structures such as nucleic acid molecules or sequences, vectors, host cells and/or polypeptides or fragments thereof, particularly interacting in such a way that detection of said structures is possible. In particular, said recognition substances can be specific nucleic acids binding to the nucleic acid molecules of the invention, but can also be antibodies, fluorescent markers, labelled carbohydrates or lipids, antisense constructs, cDNA or mRNA molecules or fragments thereof. Of course, it is also possible that the recognition substances are not proteins or nucleic acids or antibodies, but instead, antibodies directed against the same. In this event, the recognition substances can be secondary antibodies, in particular. In a special embodiment of the invention, the recognition molecules are antibodies, antibody fragments and/or an antisense constructs, e.g. RNA interference molecules. Therefore, the invention also relates to antibodies preferably binding the sequence in accordance with SEQ ID NO 1, muteins or fragments of said sequence. For example, the invention relates to polyclonal or monoclonal antibodies, including non-human and human antibodies, chimeric antibodies, humanized antibodies or fragments of antibodies obtained following immunization, via hybridoma cells or by phage display or ribosome display (Current Protocols in Immunology, John Wiley & Sons, N.Y. (1994) EP application 173-494 (Morrison); international patent application WO 86/01533; Chem. Rev. 2001, 101, 3205-3218, Ronald H. Hoess, Protein Design and Phage Display); (Hanes & Plückthun, Proc. Nat. Acad. Sci. 94, 4937-4942 (1997); Roberts & Szostak 94, 12297-12302 (1997)). Techniques for obtaining immunogenicity against the protein or polypeptide comprise conjugation to a carrier substance or carrier molecule well-known to those skilled in the art. The protein can also be used in the presence of an adjuvant for immunization. Standard ELISA or immunoassays with the protein of SEQ ID NO 1 can be used to determine the amount of antibodies. Also, the antibody (antibody fragment) can be bound to another molecule (e.g. a fluorophore) allowing improved detection of the antibody.

(v) Production method for generating the amino acid sequence preferably in accordance with SEQ ID NO 1: The invention also relates to a vector comprising a nucleic acid molecule according to the invention. Further, the invention relates to a host cell comprising the vector according to the invention.

For example, the amino acid sequence of the invention (selected from the group of SEQ ID NO 1 to SEQ ID NO 138) may concern products produced in a recombinant fashion. These products can be produced using genetic manipulations wherein the nucleic acid encoding the required product is introduced into a suitable microorganism or a suitable cell line usually by means of a plasmid or a viral vector. The nucleic acid is expressed in the cell and translated into the amino acid sequence. The desired amino acid sequence can be obtained from the cells by extraction and purification. The proteins and polypeptides according to the use of the invention can be isolated and purified by a number of processes. These include, but are not limited to, anion or cation exchange chromatography, high performance liquid chromatography (HPLC), Ni-based chromatography of His-tagged proteins, streptavidin tag-based purification. The particular method for purification depends on the particular properties of the polypeptide and host cell and is immediately obvious to a skilled specialist.

Transfection of a vector containing the amino acid sequence in accordance with SEQ ID NO 1, in particular, or one of the muteins can be effected in prokaryotic or eukaryotic cells. Cells which can be transfected include, but are not limited to, bacterial cells (such as E. coli K12 strains, streptomyces, pseudomonades, Serratia marcescens and Salmonella typhimurium), insect cells (such as Sf9 cells), including Drosophila cells, fungi such as yeasts (S. cerevisiae, S. pombe, Pichia pastoris), plant cells and mammalian cells such as thymocytes, primary cells or cell cultures such as Jurkat, CHO cells or COS cells.

Advantageously, the expression rate in E. coli BL21 (DE3) is very high (typically 20 mg of purified protein per 1 liter of bacterial culture). As a consequence of this unusual property, muteins of the amino acid sequence preferably according to SEQ ID NO 1 can also be generated, which are likewise expressed in a stable manner and exhibit a modified redox potential (see examples). Another property of the amino acid sequence according to SEQ ID NO 1 involves binding of acidic lipids such as phosphoinositols. Furthermore, the sequence in accordance with SEQ ID NO 1 can be phosphorylated in position 86 in an in vitro kinase test, and this type of phosphorylation can also take place in vivo. As a result, it is possible to contact and thereby analytically detect the domain via phospho-specific antibodies or SH2 domains or use it for purification purposes. Also, the amino acid sequences according to SEQ ID NO 1 or corresponding muteins can be precursor molecules of active molecules, i.e., such precursor molecules can be cleaved to produce active amino acid sequences according to SEQ ID NO 1 or corresponding active muteins.

Alternatively, the polypeptide preferably SEQ ID NO 1 or the corresponding muteins can be produced by in vitro translation wherein prokaryotic or eukaryotic cell extracts are used for the translation of a cDNA-read mRNA into polypeptide sequences. Another alternative of producing the polypeptide is chemical synthesis of SEQ ID NO 1, in particular.

The sequence of the preferred amino acid sequence SEQ ID NO 1 according to the invention, i.e., EKKEQKEKEK KEQEIKKKFK LTGPIQVIHL AKACCDVKGG KNELSFKQGE QIEIIRITDN PEGKWLGRTA RGSYGYIKTT AVEIDYDSLK LKKD, can be expressed e.g. as a recombinant peptide or as an untagged or e.g. His₆-tagged protein in E. coli bacteria.

The amino acid sequence can be linked e.g. with a membrane translocation sequence (MTS) on the DNA level to become cell membrane-permeable, which is preferred. In this way, protein transduction becomes possible, i.e. the amino acid sequence of the invention is advantageously capable of entering cells and tissues (Behrens et al. Curr Gene Ther. 2003 October; 3(5): 486-94; Rojas et al., Nat. Biotechnol. 16, 370-375 (1998)).

(vi) Preferred methods of producing modifications and adaptations of the amino acid sequence in accordance with SEQ ID NO 1: In a preferred embodiment of the invention the polypeptides of the invention can be modified using various methods, particularly those enabling specific or random modification. Various methods to this end are known to those skilled in the art. These methods allow specific substitution of nucleotides, thereby generating a desired amino acid sequence or mutant. In a preferred fashion, randomizing methods (e.g. error-prone PCR, use of degenerate oligonucleotides in PCR or gene synthesis, DNA shuffling, UV radiation) are used to create a variety of muteins of the amino acid sequence in accordance with SEQ ID NO 1. Amplification, cloning, ligation etc. of the resulting nucleotide sequences is effected particularly by means of molecular-biological methods.

Particularly preferred methods for the production of muteins initially produce variants of a starting DNA or RNA. The result of the above combinatorial methods implemented using means of molecular biology is a mixture (library) of different molecules which, in a preferred fashion, can be peptides (in the form of a peptide/polypeptide library), proteins or nucleic acids (in the form of a protein or nucleic acid library). Various methods of generating such mixtures have been established in the prior art. The mixture can be produced in vivo in the form of a library of different molecules, in organisms such as bacteria, or in vitro in a test tube using enzymes or isolated biological components.

For example, either the DNA or RNA in various variants is used as a basis for producing the mixture of different molecules. Depending on the technique, said variant is subsequently translated into proteins or tested in the form of RNA or DNA. To this end, selection systems well-known to those skilled in the art are employed, i.e., methods selecting the proteins or the DNA or RNA having the desired properties. In addition to phage display, the ribosome display may be mentioned in particular (Hanes & Plückthun, Proc. Nat. Acad. Sci. 94, 4937-4942 (1997); Roberts & Szostak 94, 12297-12302 (1997)). The selected molecules are accumulated in a further step and optionally analyzed in detail (sequence, redox potential, pI etc.). One preferred combinatorial method implemented using techniques of molecular biology is the phage display technology. In a preferred fashion, bacteriophages are used to this end, i.e., viruses infecting bacteria which express proteins on the surface thereof, which tolerate changes to some degree, for instance additional segments in particular sequence regions. It is mostly protein fusions of the gene III or gene VIII surface proteins that are used, so that any desired polypeptide will be expressed as fusion protein on the surface of the phage. The supply of bacteriophages, combinatorial potential of the phage display technology, development of a phage library and growth thereof, as well as screening of the desired phages have been described sufficiently in the standard literature, so that reference can be made to the corresponding literature (for example, Chem. Rev. 2001, 101, 3205-3218, Ronald H. Hoess, Protein Design and Phage Display).

Another well-known combinatorial method is combinatorial mutagenesis, in which technique the variation of the amino acids is effected indirectly on the DNA level. The process of combinatorial mutagenesis, especially the screening for activity or screening for physical properties, or the techniques of circular permutation are well-known to those skilled in the art.

Other preferred combinatorial options result from in vitro systems for the protein biosynthesis and controlled evolution, including the ribosome display technology and various techniques of DNA shuffling.

Obviously, the polypeptide library can not only be provided by using the amino acid sequence in accordance with SEQ ID NO 1. Other preferred sequences are those specified in Table 4, which contain CC motifs as part of a folded binding domain.

(vi) Methods of detecting and obtaining modifications and adaptations of the amino acid sequence in accordance with SEQ ID NO 1, which bind to target molecules: In a preferred fashion, a library of variants of the amino acid sequence in accordance with SEQ ID NO 1 is produced by means of phage display. On the basis of the elucidated structure, the residues 32, 36-43, 47, 48, 50, 59-64, 78, 80 and/or 81, preferably 32, 37, 48 and/or 81 (numbering according to the above sequence), are selected, each one in that library being substituted by all twenty natural amino acids, in principle. Having 17 residues that are varied, this corresponds to a theoretical size of the library of about 4×10²⁴ variants. It is preferred to have at least 1×10⁸ variants in the library. This library is subsequently used for screening.

Accordingly, the invention is also directed to a method making use of the peptide library according to the invention for detecting and obtaining polypeptides binding to target molecules, which method comprises the following steps:
a) immobilizing a potential target molecule on a carrier,
b) contacting peptides of the above-mentioned polypeptide library with the immobilized target molecules,
c) eliminating the polypeptides not bound to the target molecules,
d) eluting the bound polypeptides under stringent conditions, thereby obtaining the binding polypeptides.

A target molecule (e.g. a protein, a nucleic acid, a sugar or a lipid) intended to be influenced in its function is identified. Of particular interest are those target molecules playing a role in redox signalling, e.g. NF-kB, phosphatases (SHP-2), transcription factors, lipids. Also preferred are target molecules that are bound by a sequence in accordance with SEQ ID NO 1 or SEQ ID NO 2 to SEQ ID NO 138 in oxidized or reduced state only and have different fluorescence properties in the bound state compared to the non bound state. Advantageously, this allows the use of redox potential-dependent fluorescence markers in a cell.

More specifically, the target molecule is immobilized on a column and the phage library is passed through the column. If the library includes suitable variants for binding, the latter will bind to the target molecule, and all the other phages will be washed directly from the column. Subsequently, under more stringent conditions (pH value=2.2), the bound phages can also be washed from the column. In this way, a selection of the original library is obtained. The phages included are grown and passed over the column once again. This cycle is repeated 5-10 times, for example, during which process the number of phages, i.e., of the variants, becomes smaller and smaller. Finally, the phages are subjected to sequencing. In this way, it is possible to determine in detail which variants of the domain would be suitable for binding to the target molecule.

Initially, the library is screened under oxidizing/reducing conditions. Following several cycles of selection, screening is continued under opposite conditions where those phages are selected which do not bind, i.e., elute from the column immediately. In this way, variants of the domain are obtained which bind to the target molecule under reducing or oxidizing conditions only.

For example, important cellular target structures, e.g. those proteins which play a mediating role in HIV infections, can be screened by means of the molecular library according to the invention. Obviously, screening the library with molecular targets which are relevant in diseases and have been demonstrated to play an important role in redox-dependent processes are not limited to proteins mediating infections such as HIV infection, but also concerns proteins playing a crucial role in tumor formation, allergic diseases and autoimmune diseases. Examples are the transcription factors NF-kB and AP-1 stimulated in the air canals of asthmatic patients due to shifted redox balance. NF-kB is also stimulated by angiotensin IT which may cause oxidative stress e.g. in chronic renal diseases.

Being disease-relevant as well, redox-sensitive phosphatases are also preferred as target molecules. The small effect of “non-specific” antioxidants as medicaments in clinical studies is attributable to the fact that specific and sensitive biomarkers describing the redox phenotype of cardiovascular diseases are unavailable as yet.

In a preferred embodiment the method is carried out under reducing conditions, especially in the presence of dithiothreitol, or under oxidizing conditions, especially in the presence of hydrogen peroxide.

In another preferred embodiment the polypeptide library is a phage library.

In another preferred embodiment the detected binding polypeptide preferably grown from the phage library is formulated with a pharmaceutically acceptable carrier.

Accordingly, the invention also relates to a method for the production of a pharmaceutical formulation, comprising the method according to the invention, preferably in a form wherein the detected binding polypeptide is formulated with a pharmaceutically acceptable carrier, and furthermore, contacting the identified binding polypeptides with a pharmaceutically acceptable carrier.

The invention also relates to the use of the nucleic acid molecules according to the invention or of the polypeptides of the invention together with a membrane translocation sequence or a GFP construct for the identification of redox dependent protein-protein or protein-lipid interactions.

The invention also relates to a method for the detection of target molecules whose inhibition is switchable in dependence on the redox potential, comprising the steps of:
providing a sample comprising at least one target molecule candidate;
contacting one of the polypeptides of the invention or of the polypeptide library of the invention with the sample; and
detecting the interaction, particularly binding between the target molecule and the polypeptide.

In a preferred embodiment of the invention, the method is carried out in such a way that the polypeptide library is produced using a phage display method under oxidizing or reducing conditions.

In another preferred embodiment the method further comprises formulating the detected target molecule in a pharmaceutically acceptable form.

The invention also relates to the use of the detected binding polypeptide or target molecule as a pharmaceutical agent for the treatment of diseases associated with oxidative stress or a change in the redox potential of cells.

(vii) Further advantageous uses of the amino acid sequence in accordance with SEQ ID NO 1 and muteins derived therefrom: In a preferred embodiment of the invention the amino acid sequence according to SEQ ID NO 1 or muteins thereof can be used as bait proteins in a yeast two-hybrid or three-hybrid system (e.g. U.S. Pat. No. 5,283,317; Zervos et al. (1993), Cell 72: 223-232; Madura et al. (1993), J. Biol. Chem. 268: 12046-12054; Bartel et al. (1993), Biotechniques 14: 920-924; Iwabuchi et al. (1993) Oncogene 8; 1693-1696; and Brent, WO 94/10300) to identify other proteins interacting with the protein of SEQ ID NO 1 or muteins thereof, possibly modulating the activity. Such binding proteins may also be involved in processes, e.g. signal transduction in cells, wherein the peptide in accordance with SEQ ID NO 1 is involved.

Another advantageous embodiment of the invention is screening to identify modulators (e.g. antisense molecules, polypeptides, peptidomimetics, small molecules) which bind to nucleic acid sequences, polypeptides or proteins consisting of or derived from SEQ ID NO 1 or variants thereof or change the activity, expression or other properties thereof.

Another advantageous embodiment relates to an assay wherein the muteins having different redox potentials are bound to or contacted with a support material (e.g. in a plate format), the cysteines of the muteins being covalently linked with a marker molecule (e.g. a fluorophore, dye, radioactive molecule). Under conditions that are oxidizing in comparison to the cysteine-marker bond (preferably a sulfur-sulfur bond), the marker molecule preferably remains bound to the mutein. Changing the redox potential by means of a solution having a different redox potential, e.g. a cellular lysate, or by means of a solution having a different pH value or a solution having a combination of different redox potential and pH value will result in release of the marker molecule. It is only either in the free state or when bound to the peptide according to SEQ ID NO 1/mutein that the marker molecule can exhibit a particular property suitable for measuring the redox potential of the solution (e.g. cell lysate). Thus, for example, a molecule having fluorescence in the free state only can be released by incubation with a solution whose redox potential is to be determined. The share of free fluorophore is in correlation with the redox potential of the solution. However, it is also possible to remove the marker molecule released in wash steps under specific redox potential conditions and determine the amount of marker molecule still bound. By using SEQ ID NO 1 and muteins thereof, with a redox potential varying over a wide spectrum, it will be possible by means of the above procedure to determine the redox potential of a variety of solutions, e.g. cell cultures, serums, body fluids.

In another use according to the invention, a lipid modification of both cysteine residues of SEQ ID NO 1 or its muteins is carried out: Covalent linking of the redox-active cysteines with fluorescent myristoyl or palmitoyl lipids is performed as a chemical reaction. As the amino acid sequence according to SEQ ID NO 1 has affinity to negatively charged membranes by itself, this will give rise to increased binding to membranes. If the protein is introduced into cells (e.g. by microinjection), membrane recruiting can be expected. If, on the other hand, the intracellular redox potential is changed by stimulation of the cells or by chemically induced oxidative stress, the lipid group will be eliminated and the amino acid sequence according to SEQ ID NO 1 or SEQ ID NO 1 to SEQ ID NO 138, especially in accordance with the above-mentioned setup of groups of preferred sequences, will be removed from the membrane, thereby changing the fluorescence of the fluorescent lipid. Hence, the change in redox potential results in a redox-dependent change of the fluorescence on the membrane. Alternatively, and in addition to lipid modification, the protein can be fluorescence-labelled, so that changing the redox potential should result in a decrease of the fluorescence on the membrane.

In another use according to the invention, a method is provided wherein the different muteins with varying redox potentials are specifically recognized by particular antibodies or antigen-binding fragments thereof. More specifically, the method also provides antibodies which only recognize either the reduced or oxidized form of each mutein (or of SEQ ID NO 1). The methods for obtaining the antibodies or fragments thereof by means of immunization techniques or in vitro methods (e.g. phage display, ribosome display) are well known to those skilled in the art and can be carried out according to published protocols. The mutein- and redox-specific antibodies can be employed in ELISA or RIA assays or in immunoprecipitation experiments and on protein chips. In this way, it is possible to determine the redox state of SEQ ID NO 1 and of the muteins in any solution (where the redox potential is to be determined, e.g. cellular lysates), using the antibodies and subsequent detection methods (using e.g. a secondary, enzyme coupled antibody).

In another advantageous embodiment of the invention, cells are transfected with the DNA of the respective mutein (in a vector provided with regulatory sequences, or as a fusion protein). Using the detection reaction with the mutein- and redox-specific antibody, it is possible to determine the range of the redox potential, which range is given by the differences of the redox potential of the muteins and the specificity of the antibodies: one example would be reaction of an antibody with a mutein present in oxidized state, while another mutein still is predominantly detected by an antibody recognizing reduced mutein. For example, if the first mutein has a redox potential of −250 mV and the second one has a redox potential of −240 mV, the true value of the redox potential of the solution will be between −240 and −250 mV. Using microinjection or other procedures for intracellular application of the antibody, it is also possible to estimate the intracellular redox potential of cells. By linking the mutein-specific antibody with an antibody (bispecific antibody or bispecific antibody fragment) which has a different specificity and interacts e.g. with a particular cell compartment-associated antigen, it is possible to measure the redox potential in particular compartments of the cell (e.g. endoplasmic reticulum, cell nucleus, plasma membrane) in this special variant of the method.

In another advantageous embodiment the invention also provides a method of producing biologically useful agents, wherein such agents are identified starting with the analysis of structures of the amino acid sequence in accordance with SEQ ID NO 1. For example, such substructures can be epitopes for the production of antibodies, and the method comprises the development of antibodies against the epitope. The substructures may also include mutation sites changing the binding properties and the biological activity of the proteins, and the method includes the procedure of introducing such mutations. The substructure may also comprise a linking site for binding a separate chemical group, such as a peptide, a polypeptide, a solid material (beads, gels, chromatographic media, chips, plates, glass supports), a linker, and a marker (e.g. a direct marker, such as a fluorophore, or an indirect marker, such as biotin, as part of a specific binding pair).

In another use according to the invention, a method—particularly based on a structure-based analysis—is provided to introduce possible metal-coordinating amino acids in particular positions (e.g. cysteine, histidine, glutamate).

In another use of the invention, a method is provided which enables efficient assignment of the NMR resonance signals of the muteins. Especially with muteins where the three-dimensional structure has not been fundamentally changed compared to the amino acid sequence in accordance with SEQ ID NO 1, comparison with the characterized protein in accordance with SEQ ID NO 1 provides a rapid procedure of resonance assignment. Owing to said resonance assignment, it is possible to conduct an epitope mapping of binding partners of the muteins. The screening of substance libraries thus leads to an identification of the respective binding site. Based on homology modeling, it is also possible to provide a realistic representation of the binding site in the form of a three-dimensional model.

Another use according to the invention provides a method allowing the development of ligands (binding partners) and molecular scaffolds. Using the structure and NMR assignments as a basis, it is possible to elucidate the structure of a mutein-scaffold complex and identify the chemical structures which, once modified, will change the binding affinity, binding specificity, or both, between mutein and molecular scaffold, and serve as a basis for the synthesis of a ligand having modified binding affinity, binding specificity or both. “Molecular scaffold” is understood to be a core molecule allowing covalent linkage of one or more chemical groups thereto. The chemical groups comprise, but are not limited to, hydroxyl, methyl, nitro, carboxyl groups. Molecular scaffolds bind to at least one target molecule (e.g. the polypeptide in accordance with SEQ ID NO 1 and the ligands for SEQ ID NO 1 or a mutein), and the target molecule can preferably be a protein or enzyme. Advantageous properties of a scaffold comprise binding to a target molecule binding pocket, so that one or more substituents of the scaffold will bind in a binding pocket of the target molecule. Scaffolds preferably have groups which can be modified chemically, particularly by chemical synthesis, so that combinatorial libraries can be generated. Scaffolds preferably have positions allowing linkage of other chemical groups not interfering with scaffold binding to the target molecule (e.g. the protein or peptide in accordance with SEQ ID NO 1), so that the scaffold or members of the scaffold-derived library can be modified in such a way that the scaffold-target molecule complex has advantageous properties (e.g. active transport in cells or particular organs, or the option of binding the ligand to a chromatographic material for further analysis).

(viii) Advantages of the invention: Surprisingly, the amino acid sequences according to the present application, the muteins or protein recognition domains comprising the same can be used to detect or generate redox-dependent target molecules in a specific manner.

Likewise, the invention provides a method of obtaining antibodies or antigen-binding fragments thereof—directed against either the oxidized or reduced form of the protein only via animal immunization, hybridoma cell lines or phage display.

Surprisingly, the sequences and domains according to the present application or the polypeptide library members obtained using the same can be used as intra- and extracellular markers in scientific analysis, but also in diagnosis and therapy of diseases associated with oxidative stress or with a shifted redox equilibrium in cells. More specifically, cellular changes associated with pathological changes of the redox equilibrium and with oxidative modifications of proteins can be detected, on the one hand, but can also be modulated, i.e., modified in a purposeful manner.

For example, a molecular library based on the amino acid sequence of the invention or the protein recognition domain of the invention, which may also be referred to as redox scaffolds, can be generated in a first step. Thereafter, the library can be screened or browsed with a number of clinically relevant target molecules, so that the inhibition of target molecules can be detected and tested in vitro or in vivo. Based on the results obtained, the recombinant peptides or proteins isolated from the molecular library can be tested in further clinical studies for efficiency and usability in humans and developed as pharmaceutical agents. Of course, the proteins or peptides obtained can also be used as analytical tools for in vitro and in vivo analyses of cellular redox processes.

In addition to the linear sequence pattern of the amino acid sequence according to the invention, the spatial structure thereof, in particular, represents an essential aspect of the invention. The structure is the rational basis for the production of a molecular library.

The advantageous properties of the polypeptides according to the invention are the basis on which it is possible to generate a molecular library containing a multitude of 10⁸ to 10⁸ different muteins of the amino acid sequence in accordance with SEQ ID NO 1.

The teaching according to the present application is based on the knowledge that proteins or peptides play an outstanding role in all biological systems. Being cellular “executives”, they can either assume an enzymatic function, play a role as structure or supporting protein, or assume regulatory functions in a wide variety of interactions with DNA, lipids, sugar molecules, or other proteins. As a result of the great importance of proteins, it is crucial in science and clinic to have molecular markers available for the investigation of proteins. The amino acid sequences or protein recognition domains according to the present application or the members of a polypeptide library obtained by using the same can be used as said markers in a wide variety of sectors of medicine, biology or chemistry, e.g. in microscopic investigations of proteins, identification of proteins from large complexes, or as binding molecules in the protein chip technology. As is well-known, a very large number of protein-mediated processes are controlled via mechanisms depending on the redox potential inside a cell, and therefore, the amino acid sequences or protein recognition domains according to the invention can be used with advantage as molecular probes for the investigation and control of such mechanisms. Since oxidative stress is an important factor in the formation of tumors and represents an essential epigenetic factor in the process of ageing, the products according to the invention can of course be used as molecular probes in these processes as well.

The teaching according to the present application is remarkable for the following features:
Departure from conventional technologies
New field of problems
Existence of a long-unsatisfied, urgent need for the solution of the problem solved by the invention
Hitherto vain efforts in the art
Simplicity of a particular solution, especially as it replaces more complicated teachings
Development in scientific technology has proceeded in a different direction
Achievement that rationalizes development
Erroneous ideas in the art on the solution of the problem at issue (prejudice)
Technical progress, e.g. improvement, performance enhancement, lower expense, savings of time, materials, work steps, cost or raw materials difficult to obtain, enhanced reliability, elimination of flaws, superior quality, maintenance freedom, greater efficiency, higher yield, expansion of the technical scope, provision of a further means (not necessarily an improvement), creation of a second approach (not necessarily an improvement), creation of a new field, first-time solution of a problem, reserve means, alternatives, scope for rationalization, automation and miniaturization, or enrichment of the range of available drugs
fortunate choice (out of a variety of possibilities, one has been selected, the result of which has not been predictable)
Error in prior art documents
Young field of technology
Combination invention, i.e., several known elements have been combined to achieve a surprising effect
Issue of licenses
Praise in the art and/or
Economic success.

Likewise, information processing in healthy cells is regulated in a variety of ways by oxidation or reduction processes (redox equilibria) wherein redox-dependent changes take effect on the level of modifications of nucleic acids, lipids, sugars and especially proteins. More specifically, the characterization of changes in proteins—using the molecular probes according to the invention—resulting from changes in the redox potential, and the availability of reactive oxygen species (ROS) enable detection, but also modification of a variety of biologically interesting and medically relevant ROS-modified proteins. Thus, the molecular probes according to the invention are markers that both characterize and modulate the redox potential in living cells, tissues or organisms. For this reason, the peptides of the invention can be used as probes or markers in analysis to monitor redox-sensitive interactions on cell surfaces or within cells. By analyzing redox-modified proteins it is possible to detect therapeutic proteins which can prevent disease-relevant redox potential-dependent mechanisms. In particular, protein active substances capable of specifically preventing the mechanisms pathologically modified as a result of oxidation can be detected and modified with the amino acid sequences according to the present application, especially with the members of the polypeptide library. Such specific protein biologicals have been unknown in the prior art as yet. The molecular probes of the invention, i.e., the amino acid sequences or peptides of the invention, especially the polypeptide library and muteins thereof, but also the protein recognition domains as well as the functionally analogous derivatives and structurally homologous macromolecules, have many advantageous properties for use as molecular markers/probes. For example, they are highly water-soluble, thermally stable and show no tendency of aggregation. Furthermore, they are soluble in both states (oxidized and reduced) and therefore switchable in solution over their entire range of use. They are very small and therefore capable of penetrating tissues very well. Being of human origin, they can be used in humans without causing immune reactions. One particular advantage is that their redox state can be adjusted reversibly. That is, there are two different states of the amino acid sequences, peptides or proteins according to the invention which differ in their structure, thereby allowing molecular labelling or therapeutic inhibition either in reduced or oxidized state. Furthermore, it was found that the appropriate amino acid sequences can be expressed in E. coli very well. The obtained amino acid sequences of the invention can readily be isolated via a simple purification scheme.

One inventive use is covalent linkage of the amino acid sequence in accordance with SEQ ID NO 1 via a linker (peptidic or non-peptidic) with a “caged” compound containing xenon, a fluorophore, or other marker atoms or molecules which can be used as probes in biology and medicine. Being a gas, hyperpolarized xenon represents a very promising agent—already being used in medicine—for the detection of magnetic resonance signals in NMR spectroscopy and MR imaging. In the event of hyperpolarized xenon, the conformational change between oxidized and reduced states can be detected via a change in the signal of xenon which is bound either via the covalent linkage of the caged compound or directly to the protein (Spence et al., J. Am. Chem. Soc. 126, 15287-15294 (2004); Lowery et al., Angew. Chem. Int. Edn. 43, 6320-6322 (2004)). In the latter case, mutations of a hydrophobic amino acid such as methionine, leucine, valine, isoleucine, phenylalanine, tyrosine or tryptophan by alanine are implemented, and an oligo-tyrosine sequence is attached to the C terminus of the protein.

According to the use of the invention, a mutein directed to a specific target molecule (such as NF-kB, SHP-2) may provide a method wherein the mutein is introduced in particular cells and tissues or released therein. To this end, various methods are available to a person skilled in the art, e.g. embedding the mutein in liposomes or polymer materials, with subsequent cell-specific recognition (e.g. by antibody molecules) and liberation of the mutein.

The use according to the invention comprises pharmacological compositions which comprise the polypeptide SEQ ID NO 1 or a mutein or an apoprotein. Thus, for example, the poly peptide defined by SEQ ID NO 1 or one of the muteins can be mixed with a physiologically tolerable medium. Such media comprise, but are not limited to, aqueous solutions, salt solutions, polyols (e.g. glycerol, propylene glycol, liquid polyethylene glycol) and dextrose solutions. The optimum concentrations of active components can be determined empirically according to methods well-known to those skilled in the art and depend on the respective desired composition of the pharmaceutical agents. Methods for applying the exogenous polypeptide (especially SEQ ID NO 1 and muteins thereof) to the site of disease relevant treatment comprise, but are not limited to, intradermal, intramuscular, intraperitoneal, intravenous, subcutaneous, oral and intranasal application. Other methods may comprise gene therapy, reloadable biodegradable particles, viral vectors, naked DNA and polymers. The pharmaceutical formulation of the invention can also be administered as part of a combined therapy using other agents. The sequence in accordance with SEQ ID NO 1 or the muteins can also be introduced in cells via methods of gene therapy or ex vivo for expression in a vertebrate (including Homo sapiens).

The invention also relates to a kit comprising the compounds of the invention, optionally together with information concerning combining the contents of the kit. The information concerning combining the contents of the kit relates to the use of the kit in the prophylaxis and/or therapy of diseases. For example, the information may refer to a therapeutic regimen, i.e., a concrete injection or application regimen, to the dose to be administered or other things. The relevant information does not have to be included directly in the kit, but instead, the kit may also include an internet address linking the user to information in the internet which refer to a therapeutic regimen or other things.

Without intending to be limiting, the invention will be explained in more detail with reference to the following examples. Although the examples refer to SEQ ID NO 1, in particular, analogous things apply to the sequences SEQ ID NO 2 to SEQ ID NO 138.

### Protein Expression:

The sequence of the hSH3-1 domain of the human ADAP protein (SwissProt 015117, amino acids 485 to 579) was subcloned into the pET24d vector (Novagen) via the NcoI and XhoI restriction sites. The construct additionally includes the amino acid methionine at the N terminus and the amino acids leucine and glutamate and a hexa-histidine tag at the C terminus.

The plasmid with the ADAP-hSH3-1 domain was transformed into the E. coli strain BL21 (DE3) (Invitrogen). A pre culture was incubated for 12 hours at 37° C., 185 rpm, in 5 ml of LB medium containing 34 mg/ml kanamycin. Subsequently, either 1 liter of LB main culture was inoculated in such a way that an OD₆₀₀ of 0.05 was measured in the new culture (expression of non-isotope-labelled protein), or 0.5 ml of said pre-culture was diluted in 100 ml of ¹⁵N— or ¹⁵N/¹³C-labelled minimal medium. The defined minimal medium includes 60 mM K₂HPO₄, 11 mM KH₂PO₄, pH=7.4, 2 M sodium citrate, 0.8% (w/v) glucose (˜¹³C-labelled for expression of ¹³C labelled protein), 1 mM MgSO₄, 0.75 g/l NH₄Cl (¹⁵N-labelled for ¹⁵N-labelled protein) and 34 mg/ml kanamycin. When growing in LB medium, the culture at an OD₆₀₀ of 0.5 was added with 1 mM IPTG and grown for another 3 hours. Thereafter, the culture was centrifuged for 20 minutes at 4° C., 6000 rpm. The cell pellet was stored at −70° C.

When growing in minimal medium, the 100 ml culture, once having reached an OD₆₀₀ of 2.0 (after about 12 h), was diluted with another 900 ml of fresh medium and incubated at 37° C. and 185 rpm. At an OD₆₀₀ of 0.5, the cells were added with IPTG (1 mM final concentration in medium), incubated for another 4.5 hours and centrifuged for 20 minutes at 4° C., 6000 rpm. The cell pellet was stored at −70° C.

The cell pellet was resuspended in 20 ml of NiHiTrap buffer A (containing 20 mM NaPPi, pH=7.4, 500 mM NaCl, 20 mM imidazole) and subjected ten times to ultrasonication on ice for 30 seconds at a power of 120 W at maximum, allowing a rest of one minute between each ultrasonication step. Thereafter, the cell lysate was centrifuged for 30 minutes at 4° C. and 24,500 rpm. The supernatant was passed through a 0.2 μm injection filter and loaded on a NiHiTrap column (5 ml volume, Pharmacia). Following washing with 20 mM NaPPi, pH=7.4, 500 mM NaCl, 50 mM imidazole (8 column volumes), a linear gradient of 10%-100% NiHiTrap buffer B (containing 20 mM NaPPi, pH=7.4, 500 mM NaCl, 400 mM imidazole) was employed for elution, using a total of 28 column volumes. The hSH3-1 domain eluted in the fractions 26-34 at about 35% buffer B. The eluted protein was detected using SDS-PAGE and Coomassie Blue staining. The relevant fractions were combined and concentrated to a protein concentration of 1 mM using Centriprep centrifugation filters (m.w. cutoff: 3 kDa). For NMR measurements, the buffer was replaced by 50 mM NaPPi, pH=7.0, 150 mM NaCl, 0.05% NaN₃ solution. Between 0.2 and 4 mM DTT was added to convert the protein into the reduced form, and between 0.2 and 4 mM H₂O₂ was added to the protein solution for conversion into the oxidized form.

Expression of GST-hSH3-1: The amino acids 490-579 of the ADAP protein (SwissProt 015117) were cloned into the vector pGEX4-T1 using BamHI/XhoI, and the plasmid was subsequently transformed into E. coli BL21 (DE3). Expression was effected as described above for the His linked protein.

For purification, the cell pellet was resuspended in 1×PBS (140 mM NaCl, 2.7 mM KCl, 10 mM Na₂HPO₄, 1.8 mM KH₂PO₄ pH=7.3) and subjected to ultrasonication (see above), and the filtrated supernatant was loaded on a glutathione Sepharose column (GSTrap 5 ml, Pharmacia). The protein was eluted using a step gradient (5 SV 12.5%, 5 SV 25%. 2 SV 100% buffer B, 50 mM Tris, pH=8.0, 40 mM glutathione, red). The domain was eluted in the fractions 10-18 at 25% buffer B. Detection of the eluted protein was effected using SDS-PAGE and Coomassie Blue staining. The relevant fractions were combined and dialyzed against 1×PBS. To remove the GST tag, 25 mg of protein each time was incubated with 50 U of thrombin (Merck Bioscience) at 8° C. overnight. Thereafter, the sample was concentrated to 2 ml using a Vivaspin centrifuge unit (MWC 3 kDa) and separated on a gel filtration column (Superdex 75, Pharmacia). The hSH3-1 domain eluted after about 80 ml. Detection of the proteins was effected as described above. Further treatment of the domain was the same as for the His-linked protein.

### NMR Spectroscopy

To elucidate the structure of the ADAP hSH3-1 domain, a number of NMR-spectroscopic experiments were performed. All experiments were measured at a sample temperature of 300 K and a protein concentration of 0.2-1.5 mM. The spectra were obtained on a Bruker Avance 600 spectrometer with a cryo sample head or on a Bruker DMX600 spectrometer with a triple resonance sample head.

For backbone resonance assignment, a standard pair of CBCANNH/CBCACONNH spectra (Grzesiek et al., J. Magn. Res. 1992, 99, 201; Grzesiek et al., J. Am. Chem. Soc. 1992, 114, 6291) was recorded with a uniformly ¹⁵N/¹³C-labelled sample, and virtually complete backbone assignment for the oxidized and reduced forms of the ADAP hSH3-1 domain was obtained each time. The residues 1-11 and 91-102 in both forms are not visible in the spectra, which is probably due to increased flexibility and modified relaxation properties associated therewith. More specifically, the HCCH-TOCSY spectrum was used for side chain assignment. It was possible to assign more than 95% of the side chain resonances for both the reduced and oxidized forms of the protein. Based on the virtually complete assignment of the nuclear resonances of the ADAP hSH3-1 domain, the signals in the ¹⁵N NOESY HSQC, ¹³C-HMQC NOESY and ²D-¹H-NOESY-spectra were subsequently assigned, supplying distance-related information required in structure calculation (see below). The software used was TOPSPIN (Bruker, Rheinstetten, Germany); ANSIG 3.3, P. Kraulis, ANSIG: A program for the Assignment of Protein ¹H-²D-NMR spectra by Interactive Graphics, J. Magn. Reson. (1989) V24, pp. 627-633).

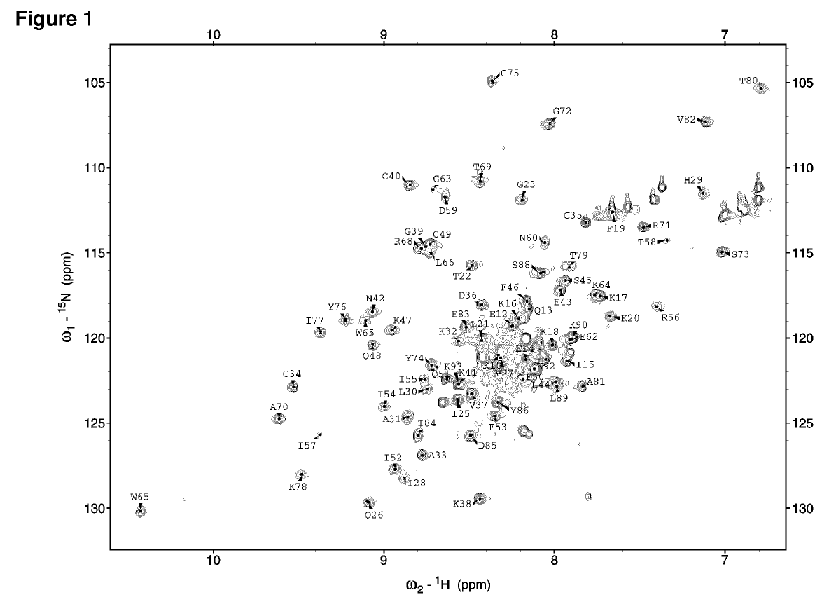

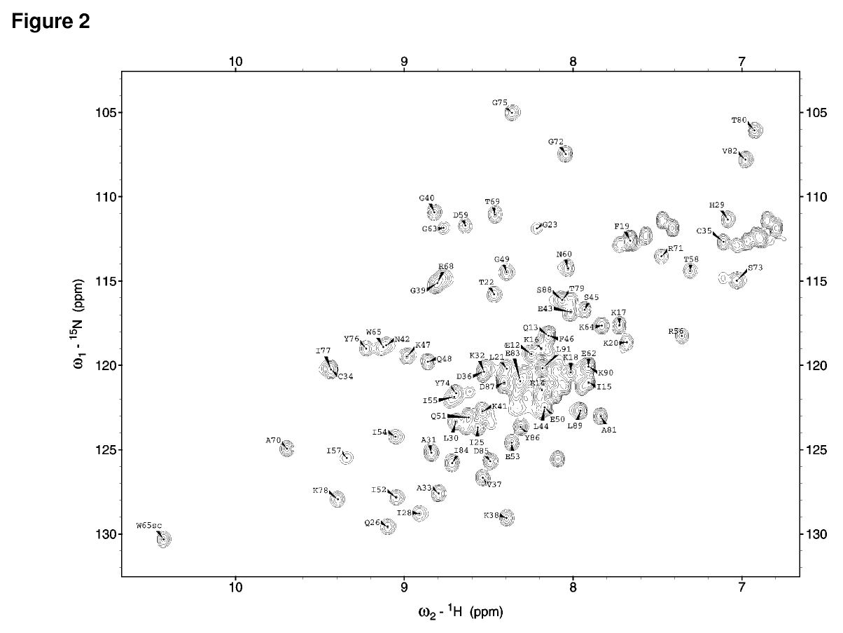

With reference to the example of a ¹⁵N-¹H correlation spectrum (¹⁵N—HSQC), the backbone assignment is illustrated in FIGS. 1 and 2. Each signal in the above two-dimensional experiment originates from a specific backbone NH group of the protein (with the exception of the NH₂ groups of asparagine and glutamine and the NH group of tryptophan). The NH group of the side chain of the tryptophan residue 65 is marked with the extension sc (side chain).

While both forms of the ADAP hSH3-1 domain show similar behavior in solution with respect to their oligomerization, the ¹⁵N—HSQC spectra of both forms show significant differences (see FIGS. 1 and 2), indicating differences in their structures as well.

The differences of the two spectra are illustrated in Fig. xy. To this end, the combined chemical shift for one residue was calculated according to formula 2 (Hajduk et al., J. Med. Chem. 1997, 40, 3144):

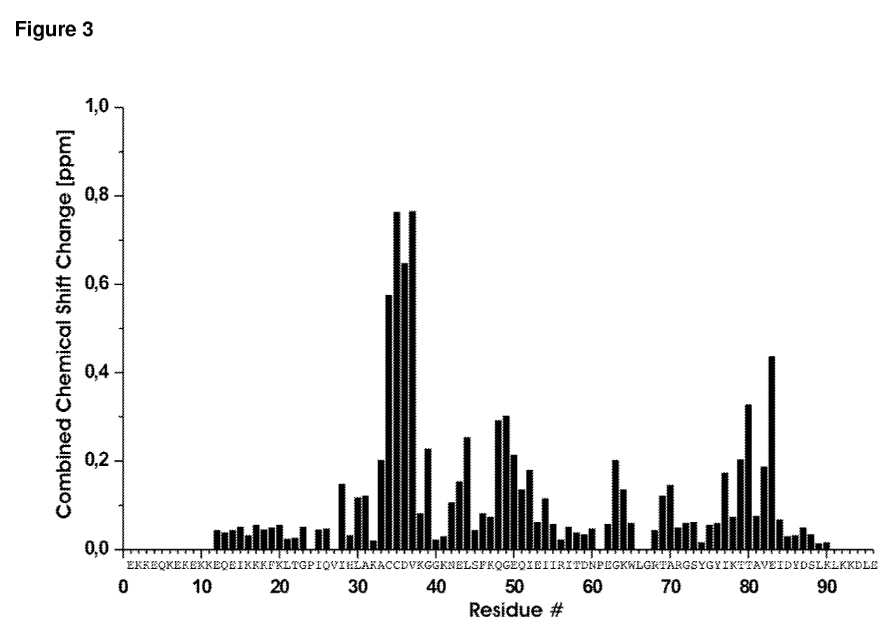

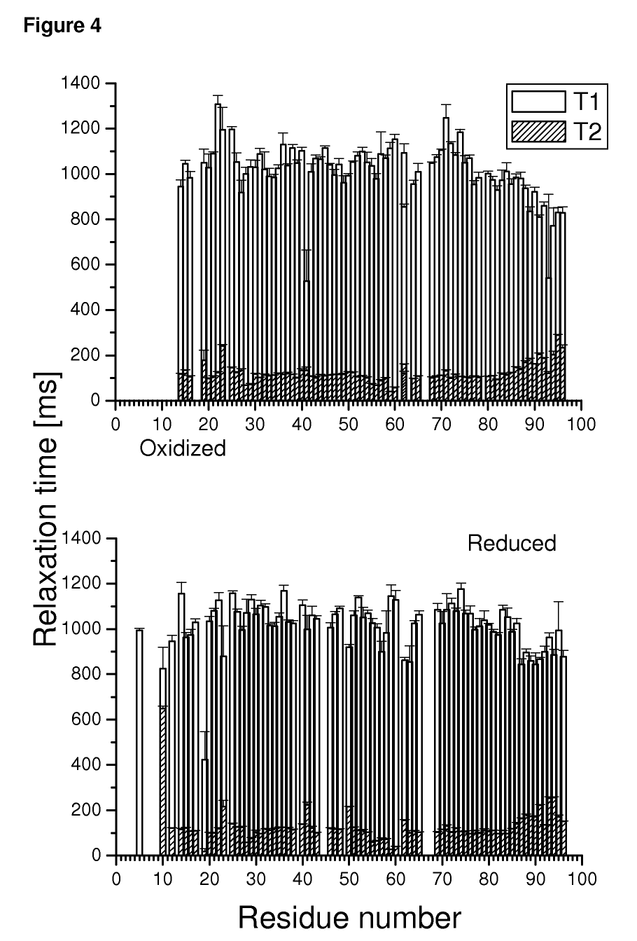

Δ=|Δ(δ¹H)|+0.2×|Δ(δ¹⁵N)|

wherein δ¹H is the chemical shift of the backbone protons, and δ¹⁵N is the chemical shift of the backbone nitrogen atoms. The most significant differences can be seen in the region around the two cysteine residues (see FIG. 3).

Based on the backbone assignment, the relaxation properties of the backbone NH groups of both forms of the protein were measured, allowing a statement as to the internal flexibility of the domain and determination of the oligomerization level of the protein under the given buffer and concentration conditions.

To this end, a series of ¹⁵N T₁ and T₂ relaxation experiments (T₁ and T₂ are characteristic relaxation times) were measured (Farrow et al., Biochemistry of 1997, 36, 2390). For measurement of T₁, the delay (an experimental parameter) in successive experiments was set to 14, 70, 140, 210, 280, 420, 560, 840, 1,260, 2,800 and 7,000 μs, and in the analogous T₂ experiments the corresponding delay was set to 6, 12, 24, 36, 48, 60, 120, 180, 240, 300 and 360 μs. The resulting spectra are similar to a ¹⁵N—HSQC. The signal intensity in each spectrum is determined for each residue of the protein and plotted versus delay.

Using regression with a singly exponential function, the T₁ and T₂ relaxation times are determined for each residue, and the values obtained are shown in FIG. 4. The Sparky program (Version 3.1, T. D. Goddard and D. G. Kneller, SPARKY 3, University of California, San Francisco) was used for regression.

The largely uniform distribution of the T₁ and T₂ relaxation times in both forms of the ADAP hSH3-1 domain allows the conclusion that the protein in each case forms a stable domain having low internal mobility.

Furthermore, the degree of oligomerization of the proteins can be derived from the values of the T₁ and T₂ relaxation times. To this end, the mean value of the T₁ and T₂ relaxation times is calculated, and the correlation time of rotation of the proteins is calculated using the formula below. The correlation time is a measure for the molecular weight of a body and therefore allows conclusions as to possible oligomerization of the proteins (Gryk et al., J. Mol. Biol. 1998, 280, 879):

$${τ}_{C} = \frac{{\{ [ 6 ( {T}_{1} / {T}_{2} ) - 7 ] \frac{1}{4} \}}^{1 / 2}}{{Ω}_{N} \cdot 2 π} \qquad ( 1 )$$

wherein T₁ and T₂ are the mean ¹⁵N relaxation times and Ω_{N} is the Larmor frequency for ¹⁵N at a given magnetic field strength (in the present case Ω_{N}=60.827865 MHz).

Table 1 shows the values for the average T₁ and T₂ relaxation times of both forms of the ADAP hSH3-1 domain and the correlation times τ_{c} calculated therefrom:

| | T₁ [ms] | T₂ [ms] | τ_{c} [ns] |
|---|---|---|---|
| Reduced | 1,010.92 | 126.44 | 8.4 |
| Oxidized | 1,012.56 | 122.39 | 8.5 |

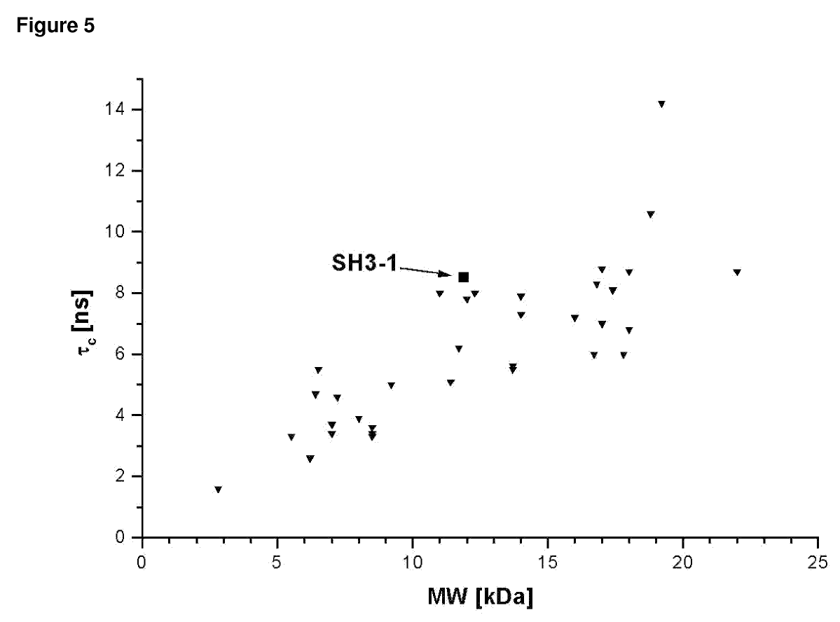

Within the scope of the measuring accuracy, both forms of the domain have equal correlation times, thus allowing the conclusion that both forms also have equal degrees of oligomerization. A comparison with correlation times of other proteins with known degree of oligomerization shows that the ADAP hSH3-1 domain is present as monomer both in the reduced and oxidized forms (see FIG. 5). This is the case even at the unphysiologically high concentrations used to perform the NMR experiments. Indeed, it is safe to say that the domain is present as monomer at low concentrations as well.

### Determination of the Redox Potential

The redox potential of the conversion of the ADAP hSH3-1 domain from the reduced into the oxidized form was determined using NMR measurement. Initially, the protein was supplied in oxidized form (concentration 0.1 mM). The standard NMR buffer (see above) was also added with 0.2 mM oxidized glutathione (GSSG), and this was gradually added with reduced glutathione (GSH). The concentrations are given in the table.

| | Exp. No. | | | | | | | | | | |
|---|---|---|---|---|---|---|---|---|---|---|---|
| | 1 | 2 | 3 | 4 | 5 | 6 | 7 | 8 | 9 | 10 | 11 |
| GSH [mM] | 0.2 | 0.5 | 1.0 | 1.5 | 2.0 | 2.5 | 3.0 | 5.0 | 7.5 | 10.0 | 15.0 |
| GSSG [mM] | 0.2 | 0.2 | 0.2 | 0.2 | 0.2 | 0.2 | 0.2 | 0.2 | 0.2 | 0.2 | 0.2 |

The intensity of suitable signals was monitored over the above series of ¹⁵N—HSQC experiments. Suitable are those residues having neither overlapping of signals in the spectra of the completely oxidized and completely reduced forms nor overlapping with signals of other residues. For this reason, the residues Cys 34, Cys 35, Gln 48, Gly 49 and Thr 80 were selected.

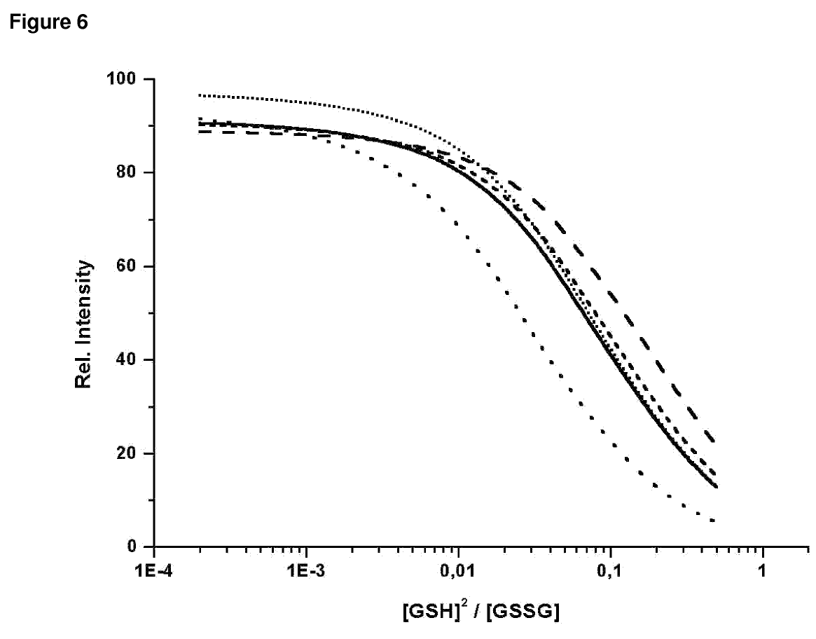

FIG. 6 shows the signal intensity profile of the above residues, plotted versus [GSH]²/[GSSG]. Each maximum intensity was arbitrarily scaled to be 100. The different residues show comparable behavior.

The regression furnishes a value of 0.05 for the inflection point of the curves, from which the redox potential of the domain can be calculated using Nernst's equation:

$$E = {E}_{O} - \frac{RT}{zF} ln \frac{{[ GSH ]}^{2}}{[ GSSG ]}$$

wherein E₀=−240 mV (standard potential for GSH/GSSG), R=8.314 JK⁻¹ mol⁻¹ (ideal gas constant), T=300 K, z=2 (number of transferred electrons), and F=9.648×10⁴ (Faraday constant). The calculated redox potential is −204 mV.

This value is in the range of an intracellular medium such as present at various stages of cell maturing. In resting cells the GSH/GSSG redox potential is about −150 mV, and the hSH3-1 domain will preferably be present in the reduced state under such conditions. In dividing cells the potential is about −200 mV, and in apoptotic cells it can even be −250 mV, in which case the hSH3-1 domain will be present in the oxidized state.

### Determination of Melting Points

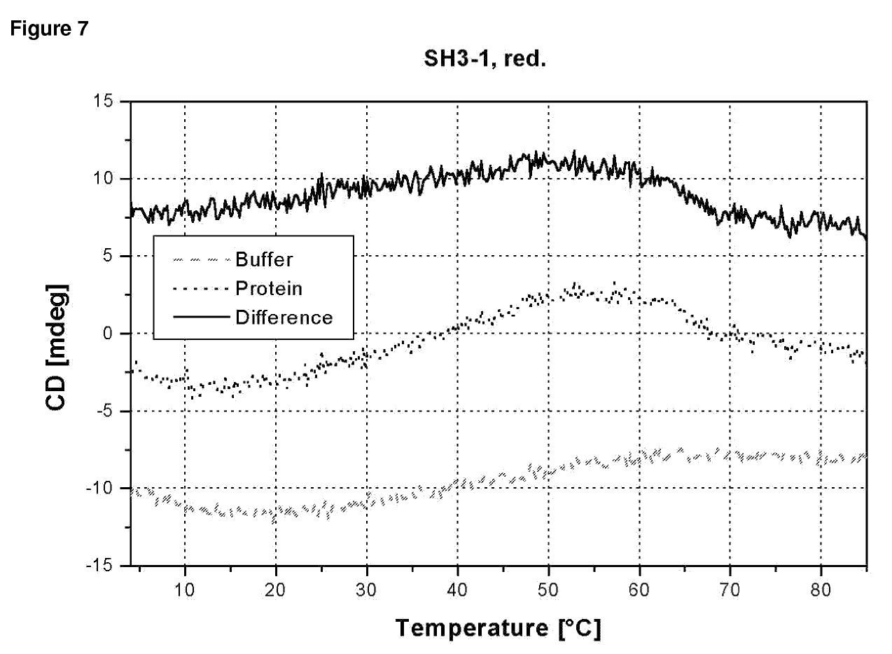

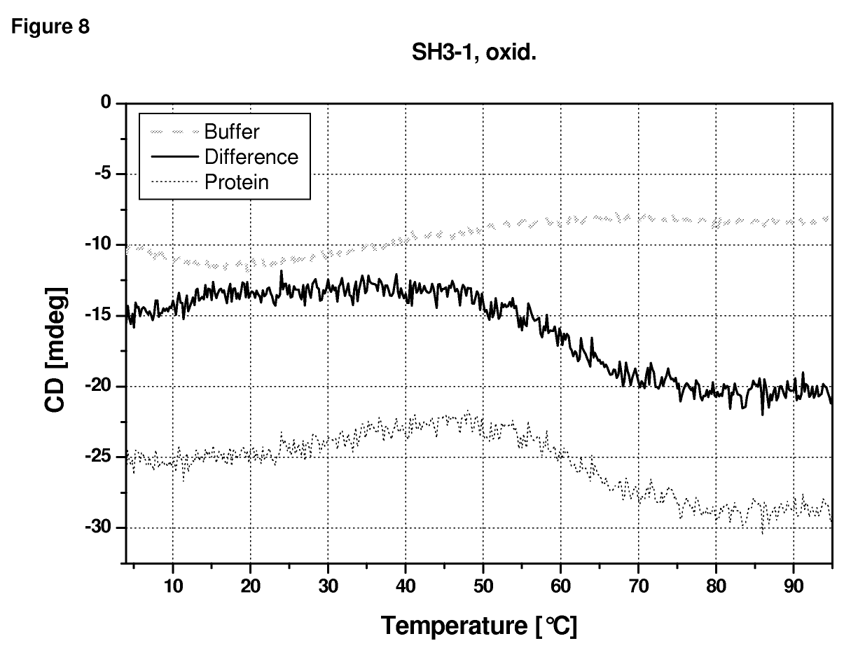

The temperature stability of the reduced and oxidized forms of the ADAP hSH3-1 domain was investigated using CD spectroscopy. To this end, samples of 15 μM in NMR buffer were placed in a CD spectrometer (Jasco 720). The CD signal was monitored at 221 nm, while the temperature of the sample was continuously increased from +4° C. to +95° C. The temperature curve of the buffer was subtracted from the curve thus obtained. The results are illustrated in FIGS. 7 and 8. Both forms of the ADAP hSH3-1 domain have a melting point of about 60° C. and thus show high thermal stability.

The structures of the reduced and oxidized forms of the ADAP hSH3-1 domain were calculated based on the evaluation of the NOESY spectra (see above). The calculations were carried out on UNIX based computers using the program CNS 1.1 (“Crystallography and NMR System (CNS): A new software system for macromolecular structure determination”. Brunger A. T., Adams P. D., Clore G. M., Delano W. L., Gros P., Grosse-Kunstleve R. W., Jiang J.-S., Kuszewski J., Nilges N., Pannu N. S., Read R. J., Rice L. M., Simosnson T., and Warren G. L. ACTA CRYST. D54, 905-921 (1998)).

The following experimental data formed the basis for calculation:
distance information based on the assignment of the NOESY spectra.
1119 distance restraints were determined for the reduced form, and 999 restraints for the oxidized form. Table 2 summarizes a specific listing and a comparison of the restraints for the two forms.
Restraints for the backbone dihedral angles φ and ψ: these restraints are based on a statistical evaluation of the chemical shifts of the Cα and Cβ atoms for the individual residues (TALOS, (Cornilescu et al., J. Biomol. NMR 1999, 13, 289)).
Hydrogen bridges: donor NH groups were determined by replacing the aqueous buffer with D₂O, acceptor oxygen atoms were identified during several cycles of structure calculation.

The structure calculation was performed using a CNS standard protocol for torsional angle dynamics. The parameters of the calculation were set as follows:

TABLE 2

| High Temperature Annealing | |
|---|---|
| Start Temperature | 50000 |
| Number of Steps | 3000 |
| Van der Waals Scale Factor | 0.1 |
| NOE Scale Factor | 50 |
| Dihedral Angle Scale Factor | 100 |
| MD Time Step | 0.015 |
| First Slow Cool Annealing | |
| Temperature | 50000 |
| Number of Steps | 20000 |
| Van der Waals Scale Factor | 1 |
| NOE Scale Factor | 50 |
| Dihedral Angle Scale Factor | 100 |
| MD Time Step | 0.015 |
| Temperature Step | 250 |
| Second Slow Cool Annealing | |
| Temperature | 3000 |
| Number of Steps | 10000 |
| Van der Waals Scale Factor | 1 |
| NOE Scale Factor | 50 |
| Dihedral Angle Scale Factor | 100 |
| MD Time Step | 0.005 |
| Temperature Step | 25 |
| Final Minimization | |
| NOE Scale Factor | 50 |
| Dihedral Angle Scale factor | 100 |
| Number of Steps | 200 |
| Number of Cycles | 10 |

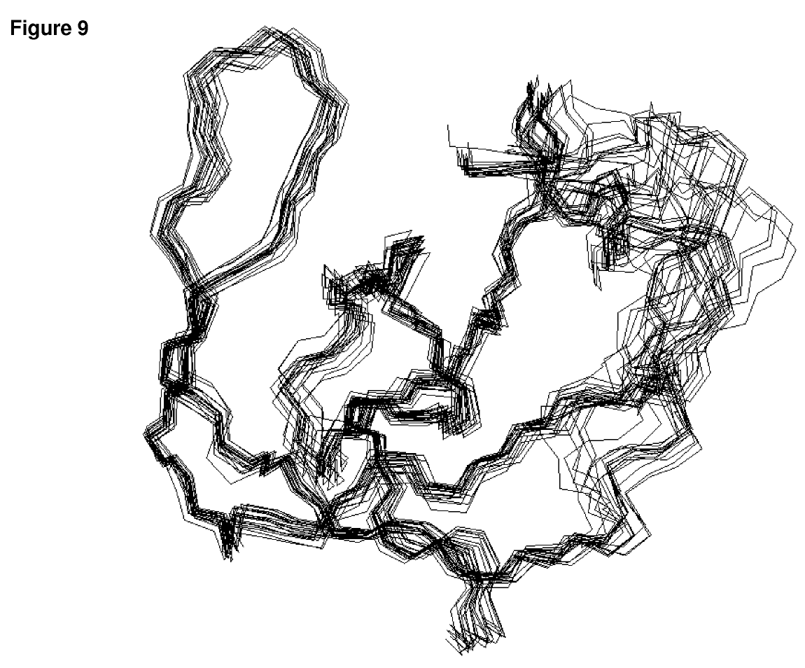

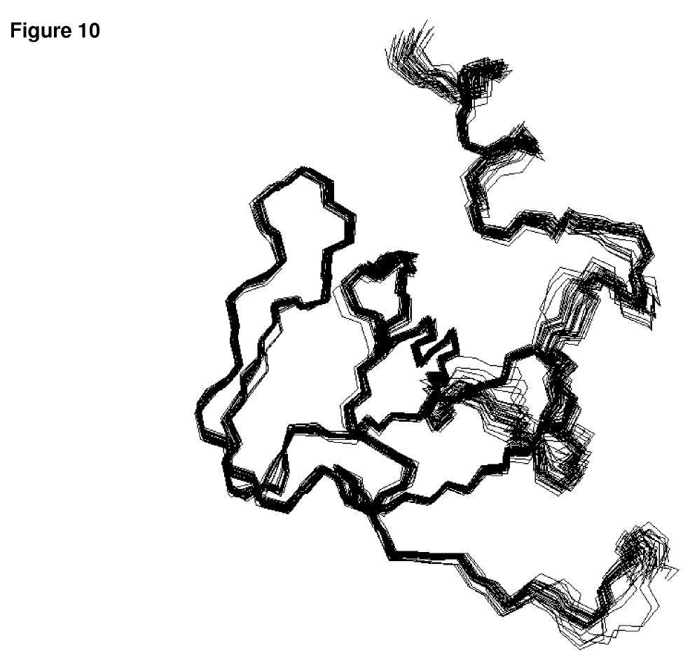

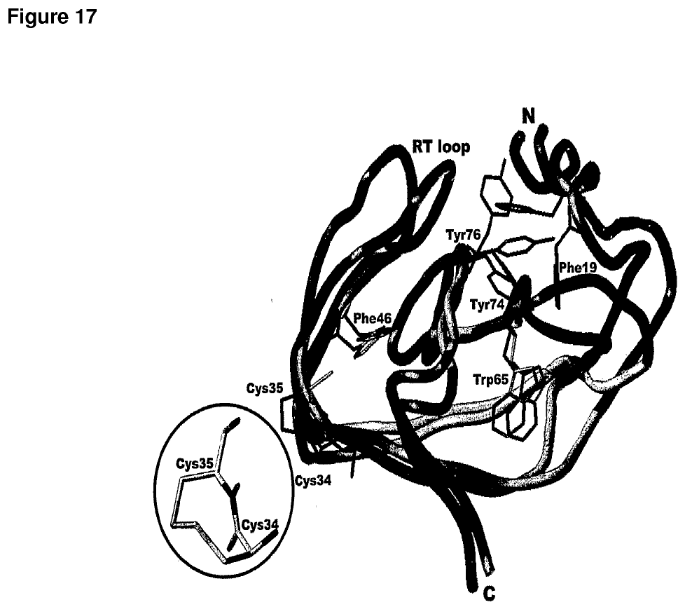

For a representation of the solution structure of the ADAP domain, 20 structures of minimum total energy were selected for each of the two forms. These ensembles are illustrated in FIGS. 9 (reduced hSH3-1) and 10 (oxidized SH3-1). A comparison of the structures is shown in FIG. 17. The structures shown are based on PDB files containing the atomic coordinates. Accordingly, a person skilled in the art can easily deduce the atomic coordinates from the Figure.

Both ensembles can be characterized in more detail as below (Table 3):

TABLE 3

| | Reduced form | Oxidized form |
|---|---|---|
| Distance restraints | | |
| Total | 1119 | 999 |
| Long-Range (>i, i + 4) | 384 | 305 |
| Dihedral angle | 97 | 110 |
| Hydrogen bridges | 0 | 0 |
| Ramachandran statistics (%) | | |
| Favored | | 78.1 |
| Additionally allowed | | 17.5 |
| Generously allowed | | 2.6 |
| Forbidden | | 1.8 |
| RMS data [Å] | | |
| Secondary structure elements | | 0.29 |
| Ordered regions | | 0.41 |
| AS 12-87 | | 0.66 |

The differences in the structure are illustrated by the following example wherein the differences (Å) between the oxidized and reduced forms were determined by superposition of two exemplary 3D structures (Table 4).

TABLE 4
Distance of the Cα atoms in the structure of reduced and
oxidized ADAP SH3-1

| Oxidized | | | | | Reduced | | | | | |
|---|---|---|---|---|---|---|---|---|---|---|
| Res. | N: | X | Y | Z | Res. | N: | X | Y | Z | Distance (Å) |
| ASP | 59 | 4.718 | 14.227 | 2.374 | ASP | 59 | −3.628 | 15.013 | −0.888 | 9 |
| PRO | 61 | 5.219 | 19.359 | 6.728 | PRO | 61 | 0.068 | 13.593 | 3.521 | 8 |
| LYS | 38 | 10.314 | 3.783 | 16.198 | LYS | 38 | 7.478 | −1.467 | 15.887 | 6 |
| ASN | 60 | 3.547 | 17.556 | 3.794 | ASN | 60 | −1.865 | 16.576 | 2.1 | 6 |
| GLY | 39 | 7.743 | 5.279 | 13.845 | GLY | 39 | 4.758 | 1.159 | 15.589 | 5 |
| CYSS | 34 | 14.52 | −2.82 | 5.742 | CYS | 34 | 15.366 | 2.097 | 6.118 | 5 |
| GLY | 40 | 5.44 | 7.312 | 16.052 | GLY | 40 | 5.213 | 3.399 | 18.605 | 5 |
| LYS | 17 | −11.66 | 4.057 | 10.311 | LYS | 17 | −10.03 | 8.14 | 8.913 | 5 |
| LYS | 18 | −8.156 | 5.232 | 11.206 | LYS | 18 | −6.264 | 8.473 | 9.309 | 4 |
| GLU | 62 | 3.244 | 17.207 | 9.166 | GLU | 62 | 0.415 | 15.559 | 6.759 | 4 |
| PHE | 19 | −6.955 | 6.479 | 7.821 | PHE | 19 | −4.661 | 8.971 | 5.896 | 4 |
| LYS | 16 | −10.43 | 2.383 | 7.126 | LYS | 16 | −9.856 | 5.905 | 5.839 | 4 |
| GLN | 13 | −8.752 | −4.062 | 9.17 | GLN | 13 | −9.912 | −0.861 | 7.684 | 4 |
| GLU | 14 | −9.02 | −1.142 | 11.593 | GLU | 14 | −9.935 | 2.038 | 10.147 | 4 |
| THR | 58 | 1.5 | 13.099 | 0.694 | THR | 58 | −0.485 | 13.324 | −2.203 | 4 |
| LYS | 41 | 1.694 | 7.051 | 16.617 | LYS | 41 | 1.568 | 4.462 | 18.626 | 3 |
| GLN | 51 | 7.759 | −5.888 | 1.891 | GLN | 51 | 10.246 | −3.776 | 1.566 | 3 |
| VAL | 37 | 12.559 | 2.127 | 13.613 | VAL | 37 | 10.018 | 0.061 | 13.504 | 3 |
| GLY | 49 | 11.941 | −6.757 | 4.97 | GLY | 49 | 14.531 | −4.824 | 4.67 | 3 |
| LYS | 11 | −6.95 | −6.966 | 3.112 | LYS | 11 | −9.459 | −6.573 | 4.932 | 3 |
| PRO | 24 | −9.715 | 1.388 | −5.236 | PRO | 24 | −8.79 | 0.018 | −2.717 | 3 |
| LYS | 32 | 10.165 | −2.331 | 1.841 | LYS | 32 | 12.182 | −0.104 | 1.692 | 3 |
| ILE | 15 | −7.374 | 0.922 | 8.854 | ILE | 15 | −7.448 | 3.718 | 7.812 | 3 |
| GLN | 48 | 13.186 | −5.308 | 8.245 | GLN | 48 | 15.076 | −3.023 | 7.962 | 3 |
| CYSS | 35 | 15.483 | −1.339 | 9.111 | CYS | 35 | 14.985 | 1.38 | 9.833 | 3 |
| THR | 80 | 12.605 | 8.453 | 6.13 | THR | 80 | 11.226 | 8.791 | 8.492 | 3 |
| GLU | 50 | 8.246 | −6.211 | 5.649 | GLU | 50 | 10.798 | −5.471 | 4.925 | 3 |
| GLY | 23 | −12.33 | 3.67 | −3.649 | GLY | 23 | −11.51 | 2.537 | −1.784 | 2 |
| LEU | 21 | −9.22 | 5.128 | 2.425 | LEU | 21 | −8.345 | 6.977 | 1.445 | 2 |
| GLY | 63 | 1.987 | 14.411 | 6.936 | GLY | 63 | 4.147 | 14.922 | 6.956 | 2 |
| LYS | 78 | 7.862 | 9.66 | 7.806 | LYS | 78 | 6.045 | 9.135 | 8.855 | 2 |
| GLU | 83 | 13.999 | 3.652 | 1.253 | GLU | 83 | 12.736 | 5.154 | 1.316 | 2 |
| ILE | 52 | 5.563 | −2.802 | 2.217 | ILE | 52 | 7.127 | −1.65 | 2.024 | 2 |
| TYR | 76 | 2.2 | 6.277 | 8.58 | TYR | 76 | 0.617 | 5.49 | 8.064 | 2 |
| THR | 79 | 9.344 | 9.217 | 4.331 | THR | 79 | 8.442 | 8.954 | 5.906 | 2 |
| LYS | 20 | −8.82 | 8.01 | 4.879 | LYS | 20 | −8.045 | 9.528 | 4.251 | 2 |
| ILE | 84 | 11.474 | 3.584 | −1.591 | ILE | 84 | 10.623 | 5.12 | −1.845 | 2 |
| LYS | 47 | 11.495 | −4.441 | 11.541 | LYS | 47 | 12.003 | −4.167 | 9.89 | 2 |
| ILE | 77 | 5.813 | 6.479 | 7.415 | ILE | 77 | 4.408 | 5.774 | 8.149 | 2 |
| ASN | 42 | −0.038 | 5.585 | 13.564 | ASN | 42 | 0.217 | 4.921 | 15.101 | 2 |
| GLY | 75 | 0.919 | 2.706 | 8.442 | GLY | 75 | 0.378 | 2.099 | 9.754 | 2 |
| ALA | 33 | 11.176 | −1.065 | 5.281 | ALA | 33 | 12.267 | −0.017 | 5.492 | 2 |
| ILE | 57 | 2.57 | 10.298 | −1.644 | ILE | 57 | 1.453 | 10.38 | −0.772 | 1 |
| ALA | 31 | 9.219 | 0.971 | 0.21 | ALA | 31 | 9.887 | 1.675 | −0.766 | 1 |
| THR | 22 | −11.75 | 5.511 | −0.387 | THR | 22 | −11.02 | 6.243 | −1.159 | 1 |
| SER | 45 | 5.635 | −0.784 | 12.861 | SER | 45 | 5.645 | −1.91 | 12.359 | 1 |
| GLN | 26 | −3.037 | 1.186 | −6.819 | GLN | 26 | −2.87 | 0.127 | −6.737 | 1 |
| ILE | 54 | 2.995 | 3.028 | −0.095 | ILE | 54 | 3.55 | 3.21 | −0.933 | 1 |
| VAL | 82 | 11.831 | 3.413 | 4.369 | VAL | 82 | 11.333 | 4.201 | 4.721 | 1 |
| SER | 73 | −1.044 | −3.403 | 7.478 | SER | 73 | −1.735 | −2.864 | 7.879 | 1 |
| PHE | 46 | 8.184 | −2.722 | 10.807 | PHE | 46 | 8.335 | −3.165 | 9.98 | 1 |
| ILE | 25 | −6.015 | 1.973 | −4.587 | ILE | 25 | −6.142 | 1.245 | −5.156 | 1 |
| ASP | 36 | 14.078 | −1.224 | 12.642 | ASP | 36 | 13.329 | −1.185 | 12.104 | 1 |
| ARG | 71 | −0.749 | −8.406 | 4.496 | ARG | 71 | −1.145 | −8.339 | 3.681 | 1 |
| GLU | 12 | −7.796 | −4.117 | 5.488 | GLU | 12 | −7.688 | −3.328 | 5.83 | 1 |
| ARG | 56 | −0.213 | 7.779 | −1.025 | ARG | 56 | −0.857 | 7.36 | −0.726 | 1 |
| GLU | 43 | 3.249 | 4.259 | 12.186 | GLU | 43 | 3.163 | 3.906 | 12.916 | 1 |
| ALA | 81 | 12.275 | 5.042 | 7.777 | ALA | 81 | 11.767 | 5.028 | 8.409 | 1 |
| LYS | 64 | 2.999 | 10.788 | 6.413 | LYS | 64 | 3.54 | 11.18 | 6.715 | 1 |
| LEU | 66 | 0.33 | 5.767 | 3.712 | LEU | 66 | 0.972 | 5.45 | 3.804 | 1 |
| GLY | 67 | 2.229 | 2.485 | 3.733 | GLY | 67 | 2.653 | 2.058 | 3.599 | 1 |
| GLU | 53 | 4.301 | −0.514 | −0.549 | GLU | 53 | 4.861 | −0.354 | −0.744 | 1 |
| GLY | 72 | −2.436 | −5.013 | 4.335 | GLY | 72 | −2.584 | −5.046 | 4.892 | 1 |
| LEU | 30 | 7.788 | −0.261 | −3.092 | LEU | 30 | 7.944 | −0.043 | −3.547 | 1 |
| TRP | 65 | 3.216 | 8.24 | 3.599 | TRP | 65 | 3.22 | 8.51 | 4.028 | 1 |
| TYR | 74 | −1.392 | 0.294 | 6.652 | TYR | 74 | −1.531 | 0.765 | 6.762 | 1 |
| ILE | 28 | 2.686 | 3.347 | −6.511 | ILE | 28 | 2.606 | 3.823 | −6.537 | 0 |
| LEU | 44 | 3.472 | 0.976 | 10.278 | LEU | 44 | 3.673 | 0.873 | 10.676 | 0 |
| ILE | 55 | −0.025 | 4.141 | −2.119 | ILE | 55 | −0.098 | 3.801 | −1.829 | 0 |
| THR | 69 | 2.748 | −4.48 | 4.279 | THR | 69 | 2.394 | −4.617 | 4.518 | 0 |
| VAL | 27 | 0.47 | 0.404 | −5.573 | VAL | 27 | 0.653 | 0.748 | −5.449 | 0 |
| ALA | 70 | 2.195 | −7.627 | 2.216 | ALA | 70 | 2.413 | −7.806 | 2.446 | 0 |
| HIS | 29 | 6.166 | 2.329 | −5.358 | HIS | 29 | 6.062 | 2.519 | −5.638 | 0 |
| ARG | 68 | 1.052 | −1.122 | 3.716 | ARG | 68 | 0.838 | −1.277 | 3.579 | 0 |

It is well-known from the literature that the backbone angle of vicinal cysteines forming an eight-membered ring via a disulfide bridge may also assume a cis conformation. In the present case, the structure calculation for the oxidized form of the hSH3-1 domain was therefore performed us ing a predefined trans as well as a cis bond. Using the same set of restraints and otherwise identical conditions of calculation, a somewhat higher total energy is obtained for the cis conformation. It is particularly in the range of the residues 33 to 36 where some NOE restraints are violated which can be satisfied by the trans conformation. For this reason, said bond is now believed to have a trans conformation.

Mutagenization of Amino Acid Residues with Respect to Molecular Libraries:

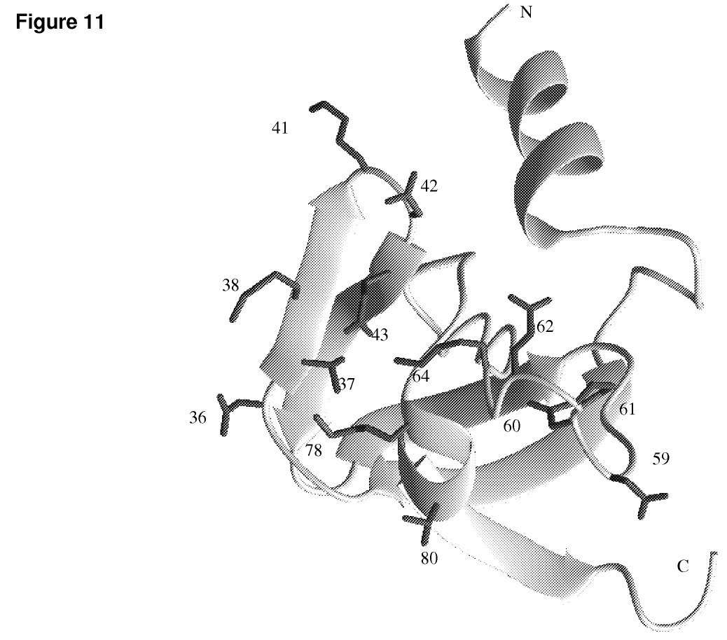

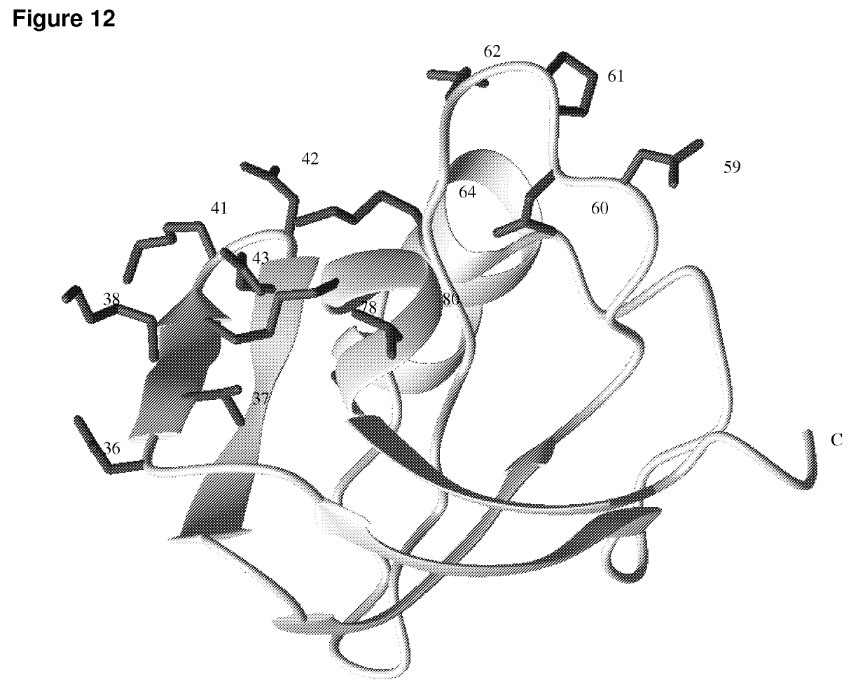

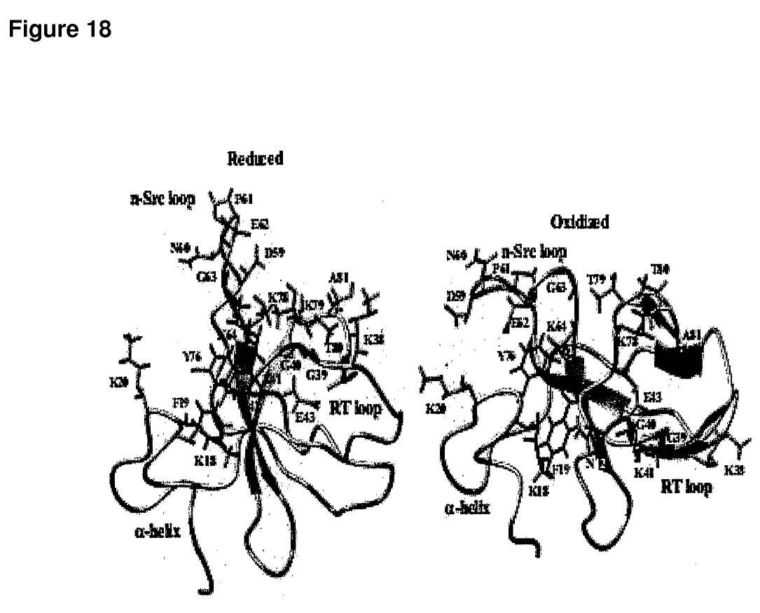

Based on the comparison of the structures, the following amino acids—among others—were selected to be subjected to mutagenesis: 18, 20, 32, 38 to 43. 59 to 64, 78, 79, 80 and/or 81 (FIGS. 11, 12 and 18). The amino acids in the above positions satisfy at least two of the following criteria:
1. They are largely exposed to solvent, i.e., in all probability, they would not prevent folding of the protein.
2. They are in relatively close spatial proximity to the pair of cysteines (<20 Å) and show a significant change in the NH group resonances between the reduced and oxidized forms. This ensures embedding of the cysteines in possible binding epitopes and, as a consequence, differentially varied behavior of the two redox forms.
3. Taken together, and considering the two redox cysteines, the above amino acids form a surface-exposed epitope with a large number of possible binding modes.

The protocol for randomization is worked out at present, and the strategy being used is as follows:

Introduction of the randomized sites by two-step gene assembly or single-step gene synthesis using a PCR. The randomized oligonucleotides are encoded by NNK triplets or by trinucleotides (N: any base possible, K: G or T) so that, on the one hand, it is possible to incorporate all 20 amino acids but, on the other hand, only the amber stop codon can appear (when using the NNK code). The latter can be suppressed in E. coli XL1Blue because TAG is read as glutamate in this strain. When using the trinucleotides, it is only a set of 19 codons (no stop codons, no cysteine) that is used.

When using phage display, the molecular library should be expressed either as gene III or gene VIII fusion protein, and the phages or phagemids are obtained according to standard protocols. More specifically, randomized PCR fragments were incorporated in the vectors pAK100 and pAK200 via SfiI restriction sites using two-fragment assembly (Krebber et al., J. Immunol. Meth. 201, 35-55 (1997)). The positions 18, 20, 32, 38 to 43, 59 to 64, 78, 79, 80 and/or 81 were randomized by NNK mutagenesis, and the corresponding PCR fragment was cloned into the vectors. One example of the construction of a library proceeds as follows:

### Primers Used for Construction of the Library:

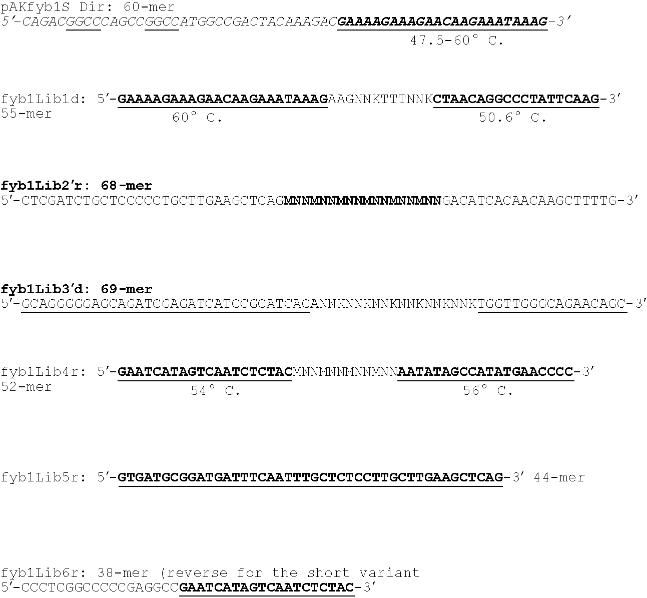

### Short Form 5Mutant (aa 494-573):

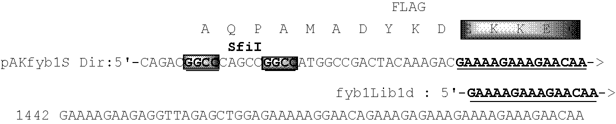

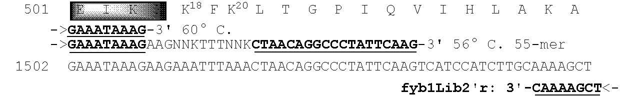

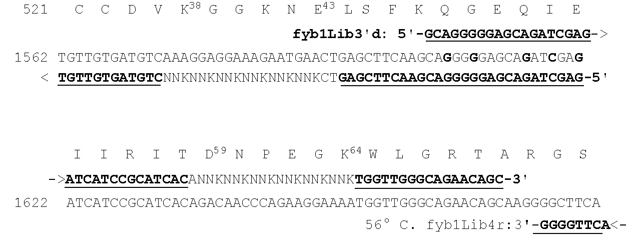

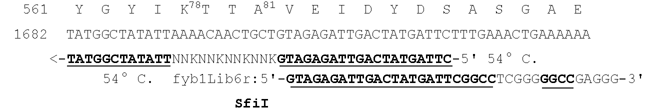

Library Construction:

### NNK Randomization:

| | | | | | | | | |
|---|---|---|---|---|---|---|---|---|
| N: | NNK | a-a | N: | NNK | a-a | N: | NNK | a-a |

| | | | | | | | | |
|---|---|---|---|---|---|---|---|---|
| 1 | AAG | K | 12 | CCT | P | 23 | GTG | V |
| 2 | AAT | N | 13 | CCC | R | 24 | GTT | V |
| 3 | ACG | T | 14 | CGT | R | 25 | TAG | — |
| 4 | ACT | T | 15 | CTG | L | 26 | TAT | Y |
| 5 | AGG | R | 16 | CTT | L | 27 | TCG | S |
| 6 | AGT | S | 17 | GAG | E | 28 | TCT | S |
| 7 | ATG | M | 18 | GAT | D | 29 | TGG | W |
| 8 | ATT | I | 19 | GCG | A | 30 | TGT | C |
| 9 | CAG | Q | 20 | GCT | A | 31 | TTG | L |
| 10 | CAT | H | 21 | GGG | G | 32 | TTT | F |
| 11 | CCG | P | 22 | GGT | G | | | |

TABLE 2
IUPAC ambiguity codes

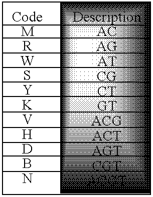

Alternative display methods are also possible, e.g. ribosome display, which is an in vitro display method wherein a significantly greater variety in the molecular library can be achieved compared to phage display (Hanes & Plückthun, Proc. Nat. Acad. Sci. 94, 4937-4942 (1997); Roberts & Szostak 94, 12297-12302 (1997)).

Similar to antibody libraries, the molecular libraries obtained will include interaction-relevant protein variants capable of recognizing a variety of different target molecules. Used as target structures are proteins, DNA, lipids, organic molecules (fluorophores, especially substances used in fluorescence microscopy) bound to a solid carrier material (either covalent coupling, or affinity based). For panning, in vitro conditions adjusting either the oxidized or reduced form of the protein will be chosen. Specifically, the following protocol will be used (using the example of a GST fusion protein): 100 mg of the GST fusion protein bound to glutathione Sepharose 4B is incubated with 10⁸ to 10¹² infectious particles at 4° C. overnight, either in the presence of 2 mM H₂O₂ or 2 mM DTT (or β-mercaptoethanol) in a volume of 400 μl. Thereafter, the batch incubated with H₂O₂ is washed several times with PBS, 0.1% Tween 20 and 2 mM H₂O₂ and subsequently eluted with PBS, 0.1% Tween 20 and 2 mM DTT (β-ME). During this step, the phages are liberated which bind to the GST target protein in the oxidized form only. Subsequently, the phages still being bound can be eluted with 100 mM glycine-HCl, pH 2.2. As to the batch incubated with DTT, washing with PBS, 0.1% Tween and 2 mM DTT (β-ME) is effected prior to eluting in PBS with 2 mM H₂O₂. In this step, only those phages are eluted this time which bind to the target protein in their oxidized form only. In the next step, the remaining binding phages are eluted with 100 mM glycine-HCl, pH 2.2. Alternatively, a “preclearing” method can be employed wherein the phages are pre-incubated under oxidizing (reducing) conditions, the GST beads with phages binding under these conditions are centrifuged and—following appropriate wash steps under reducing (oxidizing) conditions—incubated, and binding phages are subsequently eluted (after additional wash steps) with 100 mM glycine HCl, pH 2.2.

In each of the cases described above, bound phages to be further characterized are used to infect e.g. E. coli XL1Blue cells, and the phages are amplified in 2×YT medium in the presence of selection marker (kanamycin, tetracyclin or ampicillin, depending on the vector being used) at 37° C., 200 rpm overnight. After a number of such selection cycles, positive clones can be sequenced and subsequently ligated via a suitable cloning cassette into an expression vector (pTFT74, pET28d modified) for further experiments. Thereafter, target-binding proteins of the library can be expressed in E. coli or in insect cells and purified via the existing tags (His₆ tag, GST tag, Strep tag) and by means of a subsequent ion exchange or gel filtration on a column.

### Lipid Binding

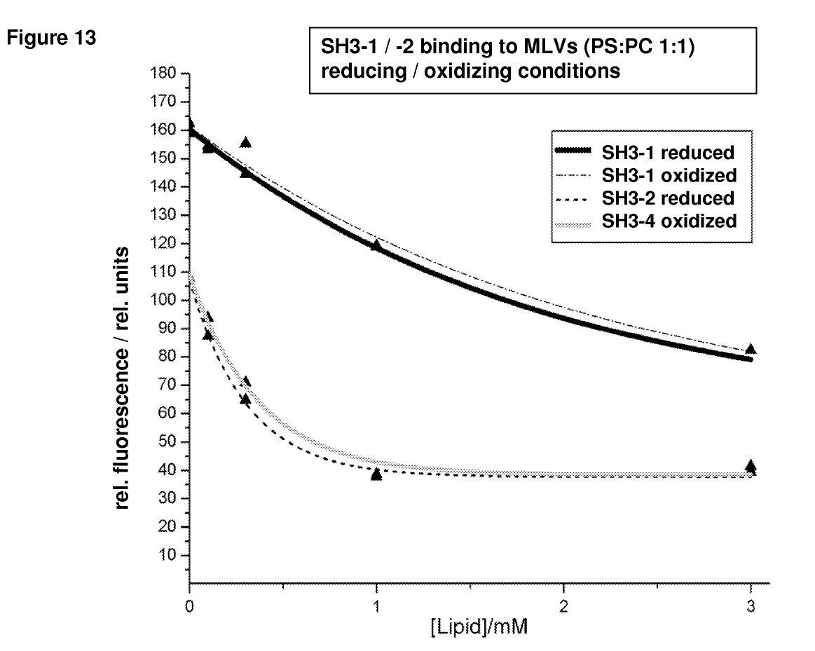

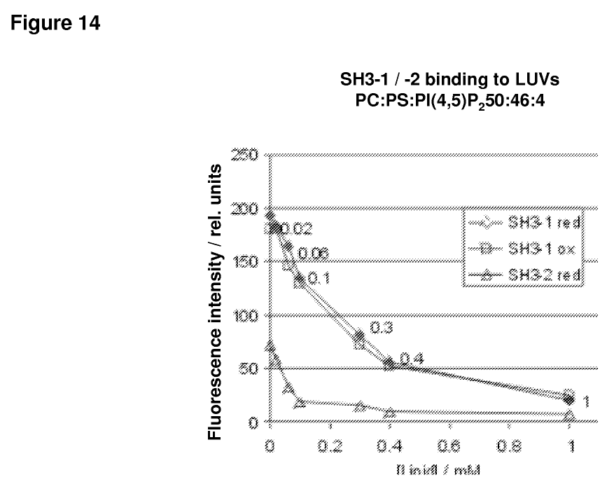

In a lipid overlay assay (Echelon) and in a liposome test the hSH3-1 domain binds to acidic lipids within lipid membranes (liposomes) (FIGS. 13, 14). In view of the structure, basic residues located on the surface of the domain (Lys15, Lys16, Lys18, Lys20, Lys38, Lys41) are at least co-responsible for binding. Structural elements probably responsible for lipid binding can also be deduced from homology studies on the hSH3-2 domain. The lipid binding properties are relevant to the invention with respect to future experiments modifying said binding function. Another possible approach is to combine protein and lipid binding properties which may represent a cooperative, possibly redox-regulated switch, in principle.

The binding properties in the sequence-homologous ADAP hSH3-2 domain were investigated. It was possible to demonstrate that the ADAP hSH3-2 domain is a lipid interaction domain (FIGS. 13, 14). It binds acidic lipids such as phosphatidyl inositols. Positively charged residues on the surface of the domain bind to polyvalent lipids such as PIP₂ or PIP₃ preferably in opposition to monovalent posphatidyl serine phospholipids. The N-terminal helix of the hSH3-2 fold is necessary for that interaction. Furthermore, basic residues of the SH3 scaffold contribute to lipid binding.

### Phosphorylations

A number of phosphorylation sites for the ADAP protein were identified in an in vitro kinase assay, including Tyr 86 (Seq. ID NO 1) in the hSH3-1 domain. The ¹⁵N—HSQC spectra of the phosphorylated and non-phosphorylated forms of the domain significantly differ from each other, implying the possibility of a structural change.

### Specific Production of Redox-Sensitive Muteins

The muteins having the mutations K32E, K32Q, V37K, Q48K and A81E were produced by site specific mutagenesis and were expressed in E. coli in analogy to SEQ ID NO 1. A few milligrams of each ¹⁵N-labelled mutein was subjected to NMR redox titration in analogy to the method described for SEQ ID NO 1. The respective redox titrations are shown in FIGS. 15 and 16. A first fit of the values for the redox potential furnishes values of −201 and −209 mV for the K32E and A81E mutant, respectively. Hence, it was possible to demonstrate that stable muteins having slightly differing redox potentials can be generated.

Other Potentially Redox-Regulated Proteins with CC Motifs

All proteins with CC motifs that were within a domain defined by the SMART domain annotation program and had no more than 4 additional cysteines were selected from the human protein data base of the SwissProt data base.

1. A polypeptide comprising
a Cys-Cys motif having a reversibly adjustable redox potential in a range of from −400 mV to +200 mV for detecting redox-dependent protein-protein or protein-lipid interactions and/or for detecting molecules which can be bound by the polypeptide in dependence on the redox potential, selected from the group consisting of the amino acid sequences in accordance with SEQ ID NO 1 to SEQ ID NO 138.

2-29. (canceled)

30. The polypeptide according to claim 1 and muteins thereof formed by substitution, deletion and/or addition of amino acids or by incorporating a glycoside residue without impairing a capability of the polypeptide of measurably interacting with a target molecule whose inhibition is switchable in dependence on the redox potential.

31. The polypeptide according to claim 1, wherein
positions 18, 20, 32, 38 to 43, 48, 50, 59 to 64, 78, 79, 80 and/or 81 of at least one amino acid residue of the polypeptide in accordance with SEQ ID NO 1 are replaced with one of the twenty natural amino acids selected from the group comprising Gly, Ala, Val, Leu, Ile, Phe, Tyr, Trp, Pro, Asp, Glu, Asn, Gln, Ser, Thr, Cys, Met, Lys, Arg or His, the polypeptide especially having a modifiable redox potential with values between −400 mV and +200 mV.

32. The polypeptide according to claim 1, wherein said polypeptide has at least 40%, preferably 60%, preferably 70%, more preferably 80%, especially preferably 90% homology to one of the polypeptide sequences in accordance with SEQ ID NO 1 to SEQ ID NO 138 and a reversibly adjustable redox potential in a range of −400 mV to +200 mV, said polypeptide being usable in the detecting redox-dependent protein-protein or protein-lipid interactions and/or in detecting molecules being bound to said polypeptides in dependence on the redox potential.

33. A polypeptide library comprising polypeptides according to claim 1.

34. A protein recognition domain comprising a polypeptide according to claim 1 or functionally analogous derivatives and structural homologues undergoing measurable interaction with a target molecule whose inhibition is switchable in dependence on the redox potential.

35. An isolated nucleic acid molecule selected from the group consisting of:
a) a nucleic acid molecule encoding an amino acid sequence according to SEQ ID NO 1 to SEQ ID NO 138, or complementary nucleotide sequences thereof,
b) a nucleic acid molecule hybridizing with a nucleotide sequence according to a) under stringent conditions,
c) a nucleic acid molecule comprising a nucleotide sequence having sufficient homology to be functionally analogous to a nucleotide sequence according to a),
d) a nucleic acid molecule which, as a consequence of the genetic code, is degenerated into a nucleotide sequence according to a), and
e) a nucleic acid molecule in accordance with a nucleotide sequence according to a), which is modified and functionally analogous to a nucleotide sequence according to a) as a result of deletions, additions, substitutions, translocations, inversions and/or insertions.

36. The nucleic acid molecule according to claim 35, wherein
the nucleotide sequence specified under c) has at least 40% homology to a nucleotide sequence specified under a).

37. The nucleic acid molecule according to claim 35, wherein
the nucleotide sequence specified under c) has at least 60%, preferably 70%, more preferably 80%, especially preferably 90% homology to a nucleotide sequence specified under a).

38. The nucleic acid molecule according to claim 35, wherein
it is a genomic DNA, a cDNA and/or an RNA.

39. A vector comprising a nucleic acid molecule according to claim 35.

40. A host cell comprising the vector according to claim 39.

41. A polypeptide encoded by a nucleic acid molecule according to claim 35.

42. (canceled)

43. A vaccine comprising a nucleic acid molecule according to claim 35, a vector comprising said nucleic acid molecule, a host comprising said nucleic acid molecule or a polypeptide encoded by said nucleic acid molecule, optionally together with a pharmaceutically tolerable carrier.

44. A method for randomized or random mutagenesis comprising providing a polypeptide according to claim 1.

45. The method of claim 44, wherein
said mutagenesis is effected using phage display, combinatorial mutagenesis, in vitro systems for protein biosynthesis, or as a controlled evolution and/or combinatorial method on a nucleic acid basis.

46. A method for detecting and/or obtaining binding polypeptides binding to target molecules, comprising the following steps:
a) immobilizing a potential target molecule on a carrier,
b) contacting peptides of the polypeptide library according to claim 33 with the immobilized target molecules,
c) eliminating the polypeptides not bound to the target molecules,
d) eluting the bound polypeptides under stringent conditions, thereby obtaining the binding polypeptides.

47. The method according to claim 46, wherein
the method is carried out under reducing conditions or under oxidizing conditions.

48. The method according to claim 46, wherein
the polypeptide library is a phage library.

49. The method according to claim 48, wherein
the detected binding polypeptides are grown from the phage library.

50. The method according to claim 46, wherein
the detected binding polypeptide is formulated with a pharmaceutically acceptable carrier.

51. A method for producing a pharmaceutical formulation, comprising the method according to claim 50 and, in addition, contacting the identified binding polypeptides with a pharmaceutically acceptable carrier.

52. Method for identifying redox-dependent protein-protein or protein-lipid interactions comprising providing a nucleic acid molecule according to any of claims 35 or a polypeptide encoded by said nucleic acid molecule together with a membrane translocation sequence or a GFP construct for identifying redox-dependent protein-protein or protein-lipid interactions.

53. A method for the detection of target molecules whose inhibition is switchable in dependence on the redox potential, comprising the steps of:
providing a sample comprising at least one target molecule candidate;
contacting a polypeptide according to any of claims 1 or a polypeptide library comprising said polypeptides with the sample; and
detecting an interaction between the target molecule and the polypeptide.

54. The method according to claim 53, wherein
the polypeptide library is produced using a phage display method under oxidizing or reducing conditions.

55. The method according to claim 53, further comprising formulating the detected target molecule in a pharmaceutically acceptable form.

56. Method for treating diseases associated with oxidative stress or a change in the redox potential of cells comprising administering the detected target molecule of claim 55 to a person in need thereof in a treating diseases associated with oxidative stress or change in the redox potential of cells effective amount.

57. A kit comprising a nucleic acid molecule according to claims 35, a vector comprising said nucleic acid molecule, a host comprising said nucleic acid molecule or a polypeptide encoded by said nucleic acid molecule and/or a vaccine comprising said nucleic acid molecule, said host or said polypeptide.

58. Method of detection of target molecules whose inhibition is switchable in dependence on the redox potential, or for the detection of polypeptides interacting with said target molecules employing the kit of claim 57.

59. A vaccine comprising a polypeptide library comprising polypeptides according to claim 1, or a protein recognition domain comprising said polypeptide or functionally analogous derivatives and structural homologues undergoing measurable interaction with a target molecule whose inhibition is switchable in dependence on the redox potential.

60. The polypeptide according to claim 31, wherein positions 32, 48 and/or 81 are replaced.

61. The method of claim 47, wherein the method is carried out in presence of dithiothreitol or in presence of hydrogen peroxide.

62. The method according to claim 53, wherein the interaction is a binding.

63. A kit comprising a polypeptide library comprising polypeptides according to claim 1, or a protein recognition domain comprising said polypeptide or functionally analogous derivatives and structural homologues undergoing measurable interaction with a target molecule whose inhibition is switchable in dependence on the redox potential and/or a vaccine comprising said polypeptide library or said protein redox domain.
